(19) United States
(12) Patent Application Publication
(10) Pub. No.: US 2010/0227327 A1
(43) Pub. Date: Sep. 9, 2010

(54) METHODS AND COMPOSITIONS FOR CONTINUOUS SINGLE-MOLECULE NUCLEIC ACID SEQUENCING BY SYNTHESIS WITH FLUOROGENIC NUCLEOTIDES

(76) Inventors: Xiaoliang Sunney Xie, Lexington, MA (US); Peter A. Sims, Cambridge, MA (US); William J. Greenleaf, Belmont, MA (US)

Correspondence Address: CLARK & ELBING LLP, 101 FEDERAL STREET, BOSTON, MA, 02110, US

(21) Appl. No.: 12/718,421

(22) Filed: Mar. 5, 2010

Related U.S. Application Data
(63) Continuation of application No. PCT/US2009/053169, filed on Aug. 7, 2009.
Application No. PCT/US2009/053169 is a continuation-in-part of application No. 12/407,486, filed on Mar. 19, 2009.
(60) Provisional application No. 61/087,445, filed on Aug. 8, 2008.
(60) Provisional application No. 61/154,674, filed on Feb. 23, 2009.

(51) Int. Cl. C12Q 1/68; C07H 19/04

(52) U.S. Cl. 435/6; 536/26.2

(57) ABSTRACT

Disclosed herein are methods and compositions for continuous single-molecule nucleic acid sequencing by synthesis with fluorogenic nucleotides.

### CROSS-REFERENCE TO RELATED APPLICATIONS

This application is a continuation of International Application No. PCT/US2009/53169, filed Aug. 7, 2009, which is a continuation-in-part of U.S. application Ser. No. 12/407,486, filed Mar. 19, 2009, and which claims benefit of U.S. Provisional Application Nos. 61/087,445, filed Aug. 8, 2008, and 61/154,674, filed Feb. 23, 2009, each of which is hereby incorporated by reference.

### STATEMENT REGARDING FEDERALLY SPONSORED RESEARCH

This invention was made with government support under OD000277 awarded by the National Institutes of Health. The government has certain rights in the invention.

### BACKGROUND OF THE INVENTION

The invention relates to the fields of single-molecule detection, single-molecule enzymology, and nucleic acid sequencing.

High-throughput, cost-effective DNA sequencing of human genomes promises to usher in a new era of personalized medicine. However, a dramatic reduction in cost and increase in speed are needed for mass-market genetic analysis to profoundly benefit human health. Single molecule sequencing methods represent the ultimate approach for miniaturization and parallelization of automated sequencing. Single molecule sequencing methods may allow for significant reduction in the cost per sequenced base, allow for significantly simpler sample preparation, and allow long read lengths. Achieving single molecule sequencing sensitivity would also allow for direct sequencing of nucleic acids (both RNA and DNA) without a prior amplification step. The elimination of this nonlinear amplification step (generally PCR) would open the door to quantitative identification of RNA transcripts from individual cells and investigation of cell-to-cell genetic sequence variability.

Most approaches to single-molecule sequencing have concentrated on either the detection of fluorescent nucleotides incorporated during DNA polymerization (Braslaysky et al. Proc. Natl. Acad. Sci. USA, 2003, 100, 3960-3964; and Harris et al. Science, 2008, 320, 106-109) or direct measurement of nucleic-acid enzyme motion (Greenleaf et al. Science, 2006, 313, 801), both of which represent so-called “sequencing-by-synthesis” techniques. While motion-based techniques appear difficult to make massively parallel, fluorescence-based methods are intrinsically parallelizable, and therefore more promising.

The use of fluorescently labeled nucleotides for single-molecule “sequencing by synthesis” (U.S. Pat. Nos. 6,911,345 and 7,033,764) is challenging because the required high concentrations of fluorescently labeled nucleotides in the reaction mixture overwhelm the signal from incorporation on a single template.

In one approach to avoid this overwhelming background signal, the four dNTPs are repeatedly flowed in and out of the sample cell, one at a time with stringent wash steps (U.S. Pat. No. 6,911,345). This approach does not allow continuous enzymatic turnovers by a single enzyme on a single template and hence reduces the speed of detection and increases costs. In addition, this method faces serious difficulty when attempting to sequence homopolymer templates, as the incorporation of many identical bases becomes difficult to detect and quantify. Moreover, the base moiety of the nucleotides is labeled with a fluorophore, which hinders subsequent polymerase reactions and must be chemically removed after each incorporation. Despite the removal of these dye labels, the synthesized DNA is still non-natural, reducing the read length of the sequencing reaction. Only short reads averaging 25-35 bases have been demonstrated with this approach, which is a serious limitation to de novo sequencing. Sanger sequencing provides the highest demonstrated, continuous read lengths for sequencing at approximately 800 bases.

Another approach circumvents the problem of short reads by the use of terminal phosphate-labeled nucleotides (U.S. Pat. No. 7,033,764). This approach allows for the release of the fluorophore upon formation of a phosphodiester bond, leaving a natural DNA. Production of natural DNA allows for the possibility of long read lengths. In order to circumvent the overwhelming background signal from the fluorescent label attached to the terminal-phosphate of nucleotides, a zero-order-wave guide is used to reduce significantly the optical probe volume (U.S. Pat. No. 7,302,146). The enzyme (and hence the DNA) is immobilized at a nanometric metal structure of the zero order wave guide. However, the small volume of the metallic structure may hinder enzymatic activity and require stringent surface chemistry treatment. Furthermore, the binding of terminal phosphate-labeled nucleotides on to the DNA template always gives rise to a signal, even if nucleotide incorporation does not occur and the nucleotide dissociates from the enzyme/nucleic acid complex. Hence, it is difficult to distinguish between nucleotide binding to the complementary strand without incorporation and actual incorporation, potentially leading to spurious signals, and therefore incorrect sequence identification. In addition, excitation of the fluorophore within the active site of the enzyme can lead to photo-induced inactivation of the polymerase (U.S. 2007/0128133).

Terminal phosphate-labeled fluorogenic nucleotides have been developed for bulk measurement applications (U.S. Pat. No. 7,041,812). These fluorogenic nucleotides are not fluorescent until hydrolysis of the label from the phosphate, providing for a background-free detection of the incorporation of the nucleotide into a nucleic acid. However, these reagents have not been employed in single-molecule detection.

Accordingly, there is a need for new methods for continuous single-molecule nucleic acid sequencing, e.g., methods with long read lengths free from the complications of enzyme immobilization and inability to distinguish nucleotide binding and incorporation events.

### SUMMARY OF THE INVENTION

In general, the invention features compositions, methods, and systems for single-molecule sequencing of nucleic acids based on the continuous measurement of the incorporation of fluorogenic nucleotides in microreactors. The invention provides numerous advantages over previous systems such as unambiguous determination of sequence, continuous sequencing, long read lengths, low overall cost, and ease of sample preparation.

In one aspect, the invention provides a method for sequencing a nucleic acid by providing a mixture in solution phase within a microreactor, which is optionally sealed, and including a single copy of a target nucleic acid, a nucleic acid replicating catalyst (e.g., DNA polymerase, RNA polymerase, ligase, RNA-dependent RNA polymerase, or reverse transcriptase), and a mixture of nucleotides that includes a first nucleotide having a first label that is substantially non-fluorescent until after incorporation of the first nucleotide into a nucleic acid based on complementarity to the target nucleic acid. The mixture in solution phase, e.g., having a volume of 0.0001 fL-1000 fL, is disposed in a microreactor, such that only one target nucleic acid is contained within the microreactor, and continuous template-dependent replication of the target nucleic acid is allowed to occur. The target nucleic acid is then sequenced by detecting in real time the individual incorporation of the first nucleotide during template-dependent replication by monitoring fluorescence emission resulting from the first label. The detection step may be repeated as desired to continue sequencing the target nucleic acid by detecting incorporation of the next nucleotide, e.g., for 10, 25, 100, 300, 1000, or 10,000 base pairs.

In certain embodiments, the mixture in solution phase further includes an activating enzyme that renders the first label fluorescent. Examples of activating enzymes include an alkaline phosphatase, acid phosphatase, galactosidase, horseradish peroxidase, phosphodiesterase, phosphotriesterase, pyruvate kinase, lactic dehydrogenase, maltose phosphorylase, glucose oxidase, lipase, and combinations thereof.

In other embodiments, the first label is photobleached after fluorescence detection. The first label may also be a phosphate label that is cleaved from the first nucleotide during incorporation.

The mixture of nucleotides may further include a second, third, and/or fourth nucleotide having a second, third, and/or fourth label that is substantially non-fluorescent until incorporation of the corresponding nucleotide into a nucleic acid based on complementarity to the target nucleic acid.

DNA or RNA may be sequenced in the methods of the invention. For DNA or RNA, a primer may be employed. Preferably, the method sequences the target nucleic acid continuously. The methods of the invention may also be multiplexed to determine the sequence of more than one target nucleotide at the same time or sequentially.

The nucleic acid in solution phase may or may not be immobilized. In certain embodiments, the nucleic acid is immobilized either to the microreactor or to a particle within the microreactor using any of a number of methods (such as biotin-streptavidin, antigen-antibody affinity, covalent attachment, or nucleic acid complementarity). For example, the nucleic acid may be attached to a micron-sized bead disposed in the microreactor or to a lid for the microreactor.

The invention further features a system for sequencing a nucleic acid that includes a plurality of microreactors each of which is capable of holding a solution phase mixture of a single copy of a target nucleic acid, a nucleic acid replicating catalyst, and a mixture of nucleotides, at least one of which has a label that is substantially non-fluorescent until after incorporation of that nucleotide into a nucleic acid based on complementarity to the target nucleic acid; and a fluorescent microscope for imaging the plurality of microreactors to sequence target nucleic acids in the microreactors by the methods described herein.

The system may further include a fluidic delivery system capable of delivering liquids to each of said plurality of microreactors and/or a light source capable of photobleaching said label after detection. This fluidic system may also be capable of purifying nucleic acids from cells for sequencing. For example, the system may be capable of isolating a single cell and purifying RNA or DNA from the cell for subsequent sequencing. In certain embodiments, the excitation source of the fluorescent microscope is capable of photobleaching the label. Microreactors may be fabricated from poly(dimethylsiloxane) (PDMS) or a combination of PDMS and glass. These devices may be coated with a fluorocarbon polymer (e.g., CYTOP) and a polyethyleneoxide-polypropyleneoxide block copolymer, such as a poloxamer (e.g., Pluronic F-108) or poloxamine. Alternatively, the reactor surface may be coated with protein-based passivation agents (e.g., bovine serum albumen or casein). PDMS microreactors may also be treated with a fluorocarbon fluid such as Fluorinert (e.g., FC-43 or FC-770). Glass surfaces may be silanized for surface passivation (e.g., 1H,1H,2H,2H-perfluorooctyltrichlorosilane or [tris(trimethylsiloxy)silylethyl]dimethylchlorosilane) and/or to allow surface conjugation of the nucleic acid or other components of the mixture (e.g., using 3-mercaptopropyltrimethoxysilane).

The invention also provides a device having a plurality of microreactors constructed in an elastomeric polymer, such as PDMS. The surfaces of the microreactors are coated with a fluorocarbon polymer, e.g., CYTOP, and a polyethyleneoxide-polypropyleneoxide block copolymer, e.g., a poloxamer or poloxamine. The elastomeric polymer is further treated with a fluorocarbon liquid, e.g., Fluorinert. The devices of the invention may also be included in a kit with one or more of a nucleic acid replicating catalyst, a mixture of nucleotides, at least one of which has a label that is substantially non-fluorescent until after incorporation of that nucleotide into a nucleic acid based on complementarity to the target nucleic acid, and an activating catalyst. Suitable additional components for these kits are described herein, including fluorogenic compounds as described herein.

The invention also features a fluorogenic compound having the formula:

Base-Sugar-Phosphate-[Self-reacting Component],

where Base is a nucleotide base, Sugar is selected from the group consisting of ribose, 2′-deoxyribose, 2′-O-methyl-ribose, ribose comprising a methylene connecting the 2′ oxygen and 4′ carbon, glycerol, 2-methyl morpholine, or threose, Phosphate is a polyphosphate (e.g., of 1-6 units), and Self-reacting Component is a moiety that undergoes an intramolecular reaction upon cleavage of the phosphate to which it is connected to form a fluorophore. In certain embodiments, Sugar is ribose or 2′-deoxyribose; Base is cytosine, guanine, adenine, thymine, uracil, xanthine, hypoxanthine, inosine, orotate, thioinosine, thiouracil, pseudouracil, 5,6-dihydrouracil, and 5-bromouracil; and/or Phosphate is a triphosphate. [Self-reacting Component] includes a self-immolative linker or a moiety that undergoes an intramolecular reaction to form a fluorophore upon removal of the phosphate.

An exemplary compound has the formula:

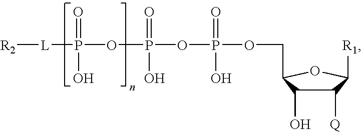

wherein Q is H, OH, or OMe, n is an integer from 1 to 4; R₁ is cytosine, guanine, adenine, thymine, or uracil; L is a self-immolative linker; and R₂ is a fluorophore bound to the linker via an amine group.

An exemplary self-immolative linker is

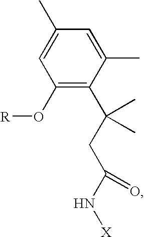

wherein R is Phosphate; and X—NH is a fluorophore bound to the linker via an amine group. In certain embodiments, X—NH has the formula

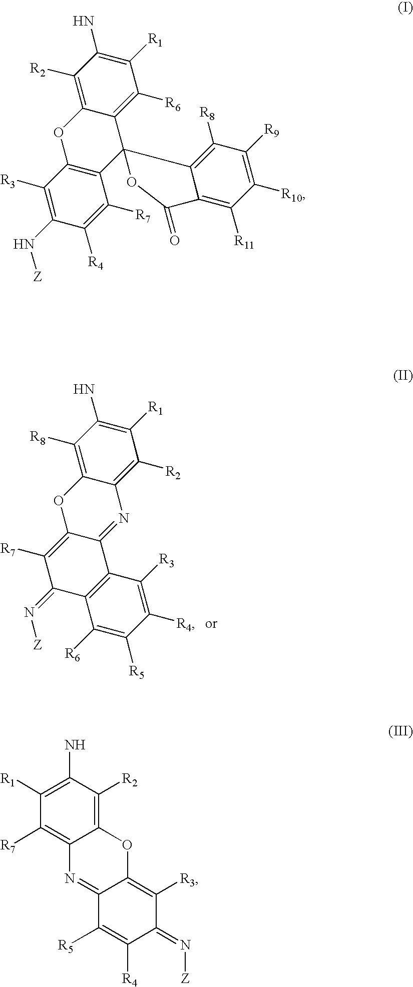

wherein each of R₁-R₁₁ is independently selected from hydrogen, halogen (i.e., F, Cl, Br, or I), sulfonate (i.e., —SO₃H), carboxy (i.e., —COOH), C₁₋₆ acyl (i.e., —COO—C₁₋₆ alkyl), or C₁₋₆ alkyl, C₁₋₆ alkoxy (i.e., —O—C₁₋₆ alkyl), C₁₋₆ alkylthio (i.e., —S—C₁₋₆ alkyl), a C₁₋₆ alkyl group interrupted with one or more heteroatoms (e.g., O, N, S, or P), C₁₋₆ haloalkyl group (e.g., perfluoro), C₃₋₆ cycloalkyl, carboxy substituted C₁₋₆ alkyl, carboxy substituted C₁₋₆ alkoxy, carboxy substituted C₁₋₆ alkylthio, C₆₋₁₀ aryl, C₄₋₉ heteroaryl (e.g., including one or more of N, O, or S and a total of 5-10 ring members), nitro, sulfonyl (i.e., —SO₂—C₁₋₆ alkyl) substituted C₁₋₆ alkyl, or hydroxyl, and each Z is independently C₁₋₆ acyl, C₁₋₆ alkyl, sulfonyl, a C₁₋₆ alkyl group interrupted with one or more heteroatoms, C₁₋₆ haloalkyl group, C₃₋₆ cycloalkyl, carboxy substituted C₁₋₆ alkyl, or sulfonyl substituted C₁₋₆ alkyl. The substituents exemplified for these fluorophores are applicable to the other fluorophores described herein.

Another compound of the invention has the formula:

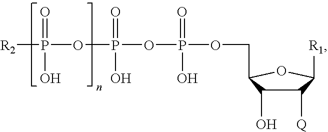

where Q is H, OH, or OMe, n is an integer from 1 to 4; R₁ is cytosine, guanine, adenine, thymine, or uracil; and R₂ is a moiety that undergoes an intramolecular reaction to form a fluorophore upon removal of the phosphate.

Examples of moieties that undergo intramolecular reactions to form a fluorophore upon removal of the phosphate have the formula:

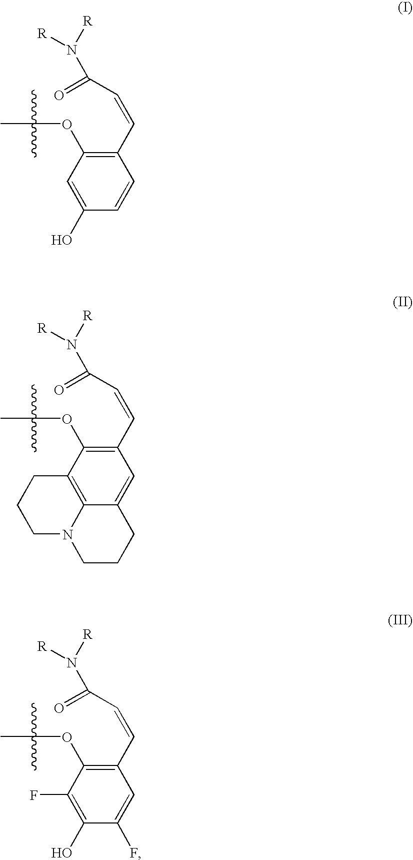

wherein each R is independently H or C₁₋₆ alkyl, or both R groups together are C₂₋₅ alkylene.

The invention further features a compound having the formula:

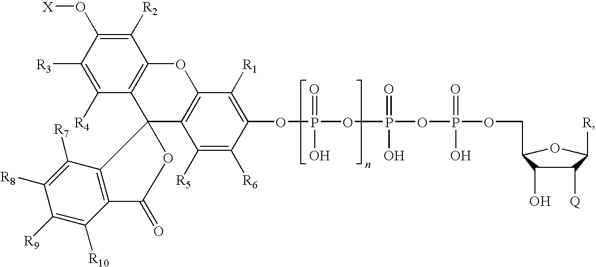

where R is a nucleotide base, Q is H, OH, or OMe, n is an integer from 1 to 4, and R₁-R₁₀ are independently selected from hydrogen, halogen, sulfonate, carboxy, C₁₋₆ acyl, or C₁₋₆ alkyl, C₁₋₆ alkoxy, C₁₋₆ alkylthio, a C₁₋₆ alkyl group interrupted with one or more heteroatoms, C₁₋₆ haloalkyl group, C₃₋₆ cycloalkyl, carboxy substituted C₁₋₆ alkyl, carboxy substituted C₁₋₆ alkoxy, carboxy substituted C₁₋₆ alkylthio, C₆₋₁₀ aryl, C₄₋₉ heteroaryl, nitro, sulfonyl substituted C₁₋₆ alkyl, or hydroxyl, and X is C₁₋₆ acyl, C₁₋₆ alkyl, sulfonyl, a C₁₋₆ alkyl group interrupted with one or more heteroatoms, C₁₋₆ haloalkyl group, C₃₋₆ cycloalkyl, carboxy substituted C₁₋₆ alkyl, or sulfonyl substituted C₁₋₆ alkyl. In certain embodiments, when R₁-R₁₀ are H, X is not C₁₋₆ alkyl, e.g., not ethyl.

Examples of this compound have the formula:

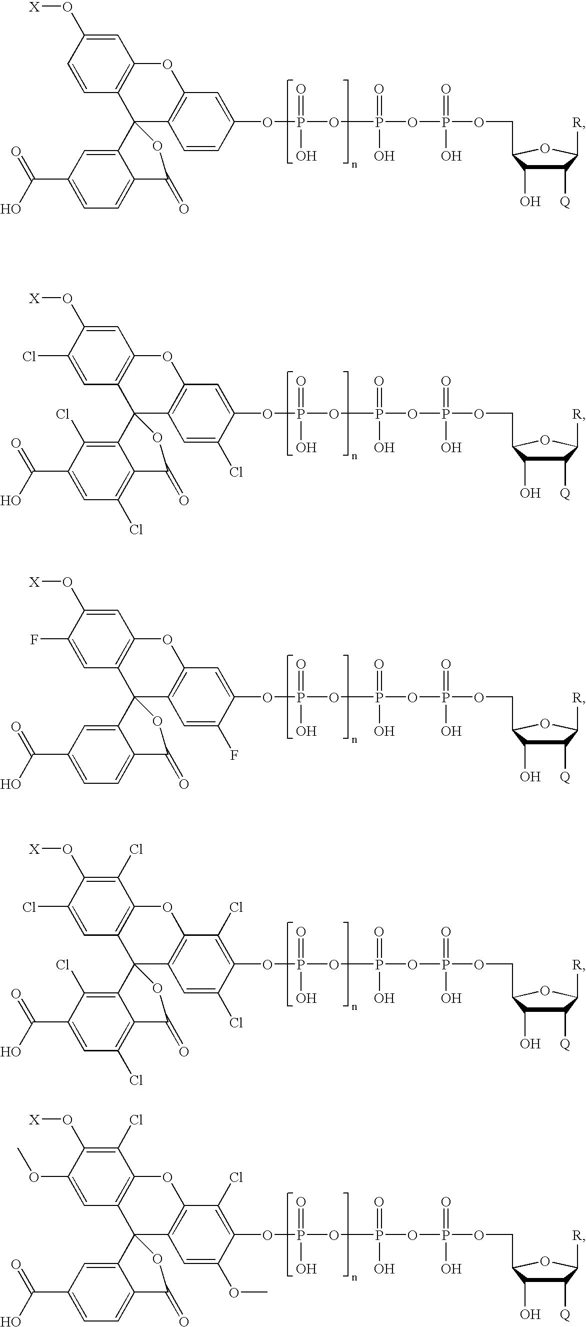

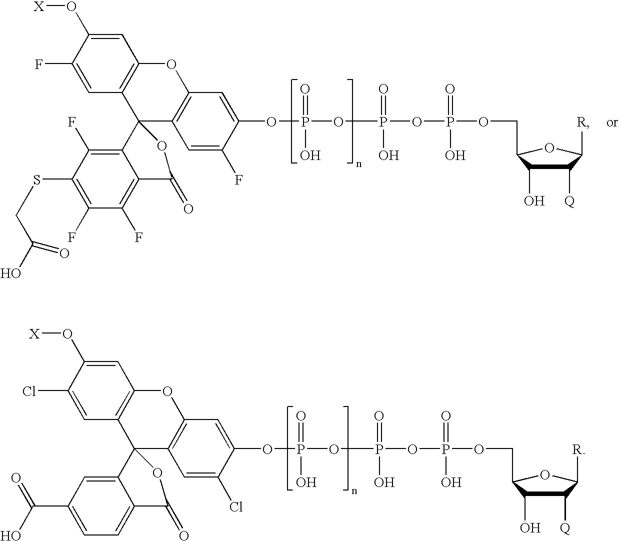

The invention further includes a compound of the formula:

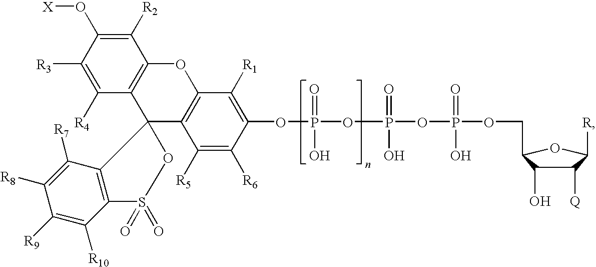

where R is a nucleotide base, Q is H, OH, or OMe, n is an integer from 1 to 4, and R₁-R₁₀ are independently selected from hydrogen, halogen, sulfonate, carboxy, C₁₋₆ acyl, or C₁₋₆ alkyl, C₁₋₆ alkoxy, C₁₋₆ alkylthio, a C₁₋₆ alkyl group interrupted with one or more heteroatoms, C₁₋₆ haloalkyl group, C₃₋₆ cycloalkyl, carboxy substituted C₁₋₆ alkyl, carboxy substituted C₁₋₆ alkoxy, carboxy substituted C₁₋₆ alkylthio, C₆₋₁₀ aryl, C₄₋₉ heteroaryl, nitro, sulfonyl substituted C₁₋₆ alkyl, or hydroxyl, and X is C₁₋₆ acyl, C₁₋₆ alkyl, sulfonyl, a C₁₋₆ alkyl group interrupted with one or more heteroatoms, C₁₋₆ haloalkyl group, C₃₋₆ cycloalkyl, carboxy substituted C₁₋₆ alkyl, or sulfonyl substituted C₁₋₆ alkyl.

Specific examples of this class of fluorogenic nucleotide substrates include:

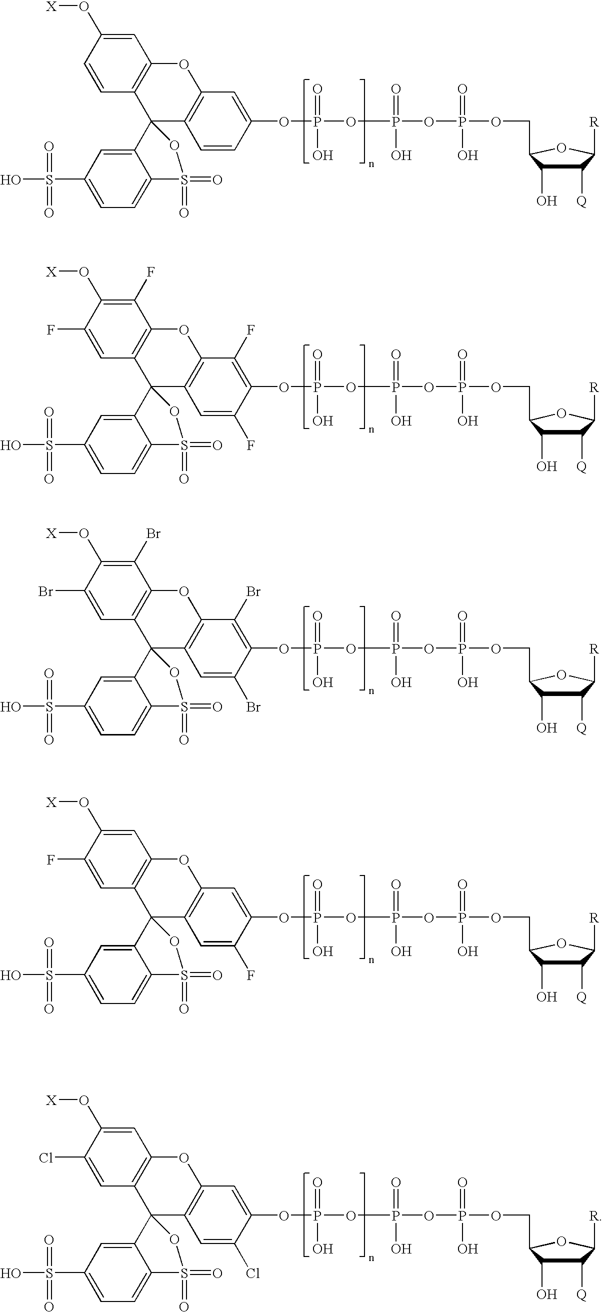

Exemplary nucleotide bases for any compound of the invention include cytosine, guanine, adenine, thymine, uracil, xanthine, hypoxanthine, inosine, orotate, thioinosine, thiouracil, pseudouracil, 5,6-dihydrouracil, and 5-bromouracil.

The invention is understood to encompass all free acid, free base, and ionic forms of the compounds, and a structure showing one such form will be interpreted as disclosing all such forms, unless otherwise noted. Appropriate counter ions may be employed for compounds in ionic form. Such counter ions include Na⁺, Cl⁻, ammonium, and trialkylammonium (e.g., triethyl or tributyl). Other counter ions are known in the art.

The invention also features kits including a nucleic acid replicating catalyst (e.g., DNA polymerase, RNA polymerase, ligase, RNA-dependent RNA polymerase, or reverse transcriptase), a mixture of nucleotides that includes a first nucleotide having a first label that is substantially non-fluorescent until after incorporation of the first nucleotide into a nucleic acid based on complementarity to the target nucleic acid (e.g., any compound of the invention), and an activating enzyme that renders the first label fluorescent (e.g., an alkaline phosphatase, acid phosphatase, galactosidase, horseradish peroxidase, phosphodiesterase, phosphotriesterase, pyruvate kinase, lactic dehydrogenase, maltose phosphorylase, glucose oxidase, lipase, or combination thereof).

By a “microreactor” is meant a vessel having a volume such that a light microscope can detect a freely diffusing fluorophore using a sensitive photon detector, e.g., capable of detecting a single molecule.

By “fluorogenic” or “substantially non-fluorescent” is meant not emitting a significant amount of fluorescence at a given wavelength until after a chemical reaction has occurred.

By “sequencing” a nucleic acid is meant identification of one or more nucleotides in, or complementary to, a target nucleic acid. Sequencing may include determination of the individual bases in sequence, determination of the presence of an oligonucleotide sequence, or determination of the class of nucleotide present, e.g., member of A-T, A-U, or G-C pair, or purine base or pyrimidine base.

By sequencing that occurs “continuously” is meant a sequencing by synthesis that results in the generation of a single complementary nucleic acid, e.g., of 10, 25, 100, 300, 1000, or 10,000 base pairs. Continuous sequencing is advantageous for determination of the number of repeats of a particular sequence. The phrase does not imply that the sequencing occurs at a constant rate. In addition, replication may occur as a result of catalysis by different copies of a catalyst, i.e., a single enzyme molecule need not catalyze synthesis of the entire complementary nucleic acid.

By “detecting in real time” is meant detecting light emitted from a label after incorporation of a labeled nucleotide into a nucleic acid but prior to incorporation of a subsequent labeled nucleotide or prior to generation of detectable signal after incorporation of a subsequent labeled nucleotide. In certain embodiments, detecting in real time occurs prior to incorporation of a subsequent labeled nucleotide.

By “incorporation” of a nucleotide into a nucleic acid is meant the formation of a chemical bond, e.g., a phosphodiester bond, between the nucleotide and another nucleotide in the nucleic acid. For example, a nucleotide may be incorporated into a replicating strand of DNA via formation of a phosphodiester bond. Other types of bonds may be formed if non-naturally occurring nucleotides are employed.

By “nucleotide” is meant a natural or synthetic ribonucleosidyl, 2′-deoxyribonucleosidyl radical, 2′-O-methyl ribonucleosidyl, Locked Nucleic Acid, peptide nucleic acid, glycerol nucleic acid, morpholino nucleic acid, or threose nucleic acid connected, e.g., via the 5′, 3′ or 2′ carbon of the radical, to a phosphate group and a base. The nucleotide may include a purine or pyrimidine base, e.g., cytosine, guanine, adenine, thymine, uracil, xanthine, hypoxanthine, inosine, orotate, thioinosine, thiouracil, pseudouracil, 5,6-dihydrouracil, and 5-bromouracil. The purine or pyrimidine may be substituted as is known in the art, e.g., with halogen (i.e., fluoro, bromo, chloro, or iodo), alkyl (e.g., methyl, ethyl, or propyl), acyl (e.g., acetyl), or amine or hydroxyl protecting groups. In certain embodiments when DNA is being sequenced, the nucleotides employed are dATP, dCTP, dGTP, and dTTP. In other embodiments when RNA is being sequenced, the nucleotides employed are ATP, CTP, GTP, and UTP. A target DNA sequence can also be sequenced with riboside bases using RNA polymerase, and a target RNA sequence can also be sequenced with deoxyriboside bases using reverse transcriptase. The term includes moieties having a single base, e.g., ATP, and moieties having multiple bases, e.g., oligonucleotides.

By “nucleotide replicating catalyst” is meant any catalyst, e.g., an enzyme, that is capable of producing a nucleic acid that is complementary to a target nucleic acid. Examples include DNA polymerases, RNA polymerases, reverse transcriptases, ligases, and RNA-dependent RNA polymerases.

Other features and advantages of the invention will be apparent from the following drawings, detailed description, and the claims.

### BRIEF DESCRIPTION OF THE DRAWINGS

FIG. 1: Fluorogenic single-molecule sequencing using a coupled enzyme assay. A) A strand of DNA with a polymerase bound, ready to add the next base to the primer strand of the DNA. Phosphates are represented by small circles, and fluorophores are represented by large circles of two different shades. Semi-transparent circles are dark because they are conjugated to one or more phosphates. B) The polymerase recognizes the correct, complementary nucleotide to add to the primer strand and binds it. C) The polymerase adds the nucleotide, generating a natural incorporated base as well as a dark fluorophore conjugated to two phosphates. D) A phosphatase cleaves one of these two phosphates, and then E) cleaves the other, generating a fluorescent molecule that can be detected.

FIG. 2: Structures of fluorescein-based fluorogenic nucleotide substrates for single molecule nucleic acid sequencing where R₁ is a nucleotide base, R₂ is a blocking group designed to minimize the absorption and fluorescence emission of the fluorogenic substrate, and n is an integer between 0 and 4. A) Substrate based on 6-carboxyfluorescein (6-FAM). B) Substrate based on 6-carboxyhexachlorofluorescein (6-HEX). C) Substrate based on 6-carboxytetrachlorofluorescein (6-TET). D) Substrate based on 6-carboxy-4′,5′-dichloro-2′,7′-dimethoxyfluorescein (6-JOE). E) Substrate based on Oregon Green™ 488. F) Substrate based on Oregon Green™ 514. G) Substrate based on 2,7-dichlorofluorescein.

FIG. 3. Structures of coumarin-generating fluorogenic nucleotide substrates for single molecule nucleic acid sequencing where R is a nucleotide base A) Substrate based on 7-hydroxycoumarin. B) Substrate based on coumarin 102. C) Substrate based on 6,8-difluoroumbelliferone.

FIG. 4: Microreactor fabrication procedure. Polystyrene beads are close-packed onto a flat glass surface. Polydimethylsiloxane (PDMS) is poured and cured onto these beads and then removed. The impregnated beads are removed mechanically, and the coupled-enzyme reaction mixture is placed between the patterned PDMS and a PDMS-coated coverslip. Upon application of pressure, sealed microreactors are formed and can be imaged from below with a light microscope.

FIG. 5: Demonstration of homogeneous fluorogenic assay for DNA polymerase activity in PDMS microreactors. A) Bright field transmission image (left) of 5 μm diameter microreactors one of which contains a polystyrene bead coated with ˜100 DNA molecules and fluorescence image (right) of the same field-of-view 5 minutes after sealing the poly-C-DNA template-coated bead, φ29 (exo-) DNA polymerase, dGTP-γ-resorufin substrate, and shrimp alkaline phosphatase (SAP). B) Bright field transmission image (left) of 1.5 μm diameter microreactors two of which contain polystyrene beads coated with ˜100 DNA molecules and fluorescence image (right) of the same field-of-view 3 minutes after sealing the poly-C-DNA template-coated beads, Klenow fragment (exo-) DNA polymerase, dGTP-γ-resorufin substrate, and SAP. One of the two microreactors contains more than one bead, and the corresponding fluorescence signal is considerably higher.

FIG. 6: Valve-based sealing of PDMS microreactors. The PDMS microreactor includes a control layer (A) which allowed for reversible sealing of the reaction chambers upon application of pressure (B).

FIG. 7: Microreactors filled with fluorescent DDAO (left is the bright-field image, and right is the fluorescence image). These reactors were generated from 1.5-micron polystyrene beads.

FIG. 8: Optics diagram for two-color total internal reflection fluorescence (TIRF) microscope, which can also be used in epifluorescence mode. A beam of 300 mW of 560 nm laser light is expanded using lenses L1 and L2 and finally focused with lens L3. This beam is directed off a dichroic beam splitter (DCBS1) and impinges on the back aperture of a 1.45 NA 60×TIR objective. Fluorescence from the sample is collected by the objective, passes through DCBS1, and is imaged with the tube lens L4. The iris defines the relevant field of view, and the DCBS2 splits fluorescence generated by resorufin from fluorescence generated by DDAO into two separate channels that are then imaged using lenses 5 and 6. The images are offset laterally slightly, then recombined by DCBS3, and imaged on an electron multiplied charged coupled device (EMCCD) camera.

FIG. 9: Detection of single resorufin molecules in PDMS microreactors. The leftmost image shows the bright-field image of microreactors generated from 1.5-micron beads. Moving to the right, the images show the DNA polymerase-based generation of a resorufin molecule in one microreactor and subsequent photobleaching. Sample is exposed to laser light for 25 ms every 1-second integration time. The molecule photobleaches after one frame.

FIG. 10: Fluorescence trajectory detected from a single microreactor in a two color sequencing experiment. One trajectory is DDAO fluorescence, and one trajectory is resorufin fluorescence. The target, template DNA sequence is (TTTATTA)ₙ. In this case resorufin labels the dTTP, and DDAO labels the dATP.

FIG. 11: A: Chemical structure of CYTOP; B: Chemical structure of Pluronic F-108; C: Schematic for non-protein, self-assembled blocking; and D: Labeled molecules of streptavidin diffusing freely in microreactors.

FIG. 12: Schematic depiction of photolithographic fabrication of microreactors in PDMS.

FIG. 13: A and B: Resorufin diffusion over 100 seconds; C and D: DDAO diffusion over 100 seconds.

FIG. 14: Structures of DDAO and 6-sulfo-DDAO.

FIG. 15: Sulfo-DDAO diffusion in PDMS after 600 seconds.

FIG. 16: Elimination of DDAO dye diffusion in CYTOP-coated PDMS over 100 seconds.

### DETAILED DESCRIPTION OF THE INVENTION

We have developed compositions and methods for detecting the synthesis of a single nucleic acid using fluorogenic nucleotides that are substrates for nucleic acid replicating catalysts and that become able to emit light as a result of incorporation of the nucleotide into a nucleic acid. The invention employs microreactors to contain the sequencing reaction. This invention overcomes limitations of previously proposed techniques for single molecule synthesis and sequencing.

Advantages of the present invention include:

1) Use of fluorogenic substrates eliminates background from unincorporated labeled nucleotides.

2) Confinement of an isolated single nucleic acid, the reaction of which can be followed continuously allowing unambiguous determination of sequence.

3) Restriction of the diffusion of the generated fluorescent label to a volume that is sufficiently small such that a single molecule can be detected above the background, e.g., Raman or autofluorescence.

4) Reduction of fluorescence signal from autohydrolysis of the fluorogenic substrate in the absence of incorporation of the labeled nucleotide.

5) Allows for a regular, dense array of microreactors enabling high-throughput, parallel nucleic acid sequencing.

6) Reduction in the amount and the cost of reagents (enzyme, labeled nucleotide, nucleic acid, etc.) required for high-throughput sequencing.

7) Confinement makes surface immobilization of the replicating catalyst unnecessary, avoiding perturbation of enzyme activities.

8) No change in the reactants is necessary during sequencing.

9) Continuous sequencing of thousands of nucleotides is possible, in principle.

10) No linear or nonlinear amplification is necessary, simplifying sample preparation.

11) The sample can be loaded quickly into the microreactors.

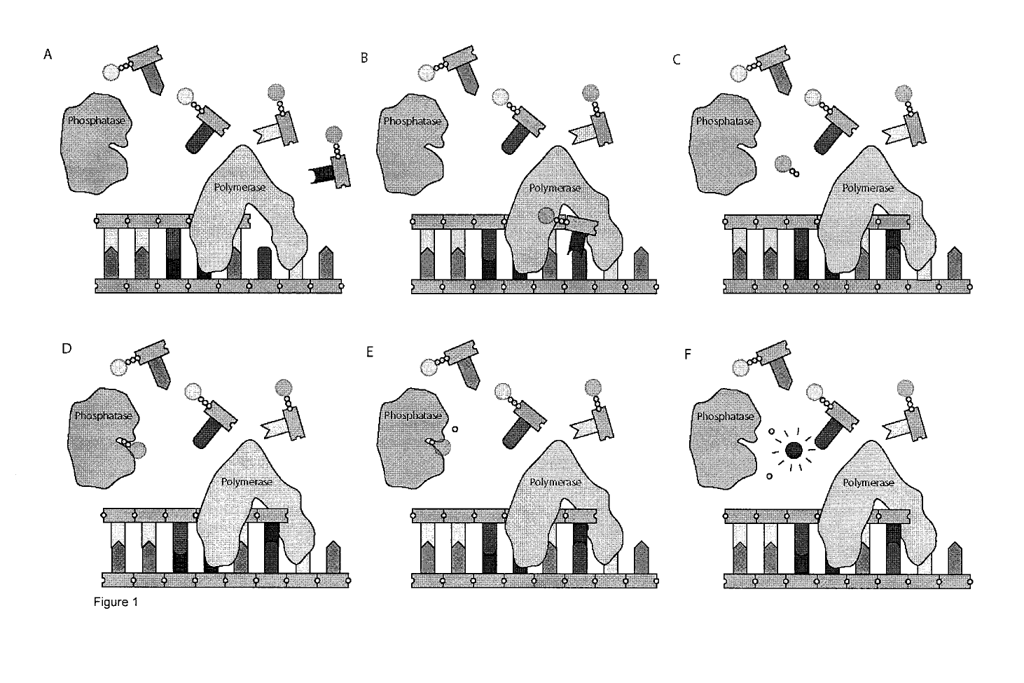

The methods are employed in connection with sequencing by synthesis, in which the incorporation of an individual nucleotide, e.g., including a single base or multiple bases, into a nucleic acid during replication is detected. As nucleotides are incorporated into a nucleic acid that is complementary to the target nucleic acid, the label is rendered able to emit light, e.g., by cleavage from the incorporated nucleotide (e.g., when bound to the terminal phosphate of a nucleotide) (FIG. 1). Because nucleotides are incorporated sequentially during replication, the incorporation of an individual nucleotide can be measured in real time as a result of the emitted light. Preferably, the label is substantially non-emitting when diffusing free in solution to reduce background that could interfere with real time detection of incorporation. Tens of thousands of bases on a single nucleic acid can be read continuously with high speeds up to 10-100 bp/sec, the technique can easily distinguish incorporation from false binding, i.e., temporary hybridization not resulting in bond formation, and no zero-order waveguide is required.

Incorporation typically results in the cleavage of a portion of the nucleotide, e.g., pyrophosphate, and the label is typically bound to the cleaved portion, i.e., does not form part of the nucleic acid after incorporation. The label may not be immediately fluorescent upon cleavage from the nucleotide. In these embodiments, chemical modification of the label or groups pendant on the label must first occur. For example, certain dyes are non-fluorescent when conjugated to a phosphate group; removal of the phosphate group, e.g., via a phosphatase, then renders the label fluorescent. Other chemical mechanisms that may be involved include acid and base catalyzed reactions and other catalytic processes described herein. Labels may alternatively become able to emit merely as a result of cleavage from the growing nucleic acid. For example, a label may be quenched or otherwise rendered non-emitting by proximity to the nitrogenous base of a nucleotide or a moiety associated with the base.

Preferably, the rate of generation of a fluorophore is more rapid than incorporation of a nucleotide into a nucleic acid. For high fidelity, rapid nucleic acid sequencing, the generation of a fluorophore is typically closely coupled in time with the incorporation of a fluorogenic label into a nucleic acid, by a nucleic acid replicating catalyst. Therefore, any activating catalyst (e.g., alkaline phosphatase) preferably acts rapidly on the fluorogenic label, yielding a fluorophore. For sequencing, the nucleic acid replicating catalyst preferably incorporates the fluorogenic label at a rate much slower than the rate at which an activating catalyst converts the fluorogenic-label to a fluorophore, so that sequentially released fluorogenic-labels are sequentially catalyzed by the activating catalyst, thereby reproducing the temporal order of nucleotide addition. In a preferred embodiment, nucleotides are incorporated by the nucleic acid replicating catalyst at a rate of approximately 1 per second, which allows rapid generation of the fluorophore, optical excitation and detection of the fluorophore, and subsequent bleaching (see, e.g., U.S. Pat. Nos. 7,125,671 and 7,041,812).

When each nucleotide is added to the synthesized strand, the nucleotide added is preferably identified. One method of determining the identity of a particular nucleotide is to attach a different, distinguishable label to each nucleotide being added, typically A, T, C, and G, or A, U, C, and G. By detecting which of the labels is added at a given point in synthesis, the corresponding nucleotide added can be identified, and, when present, the sequence of a target nucleic acid can be determined, by virtue of its complementary nature. Methods for detecting four or more optically distinguishable labels are well known in the art.

Alternatively, fewer labels may be employed. Two labels may be employed when a target double stranded nucleic acid or a single stranded nucleic acid and its complement are sequenced. In this example, one of A and T (or U) is labeled, and one of C and G is labeled. Another example of two-label detection is to label one nucleotide with a first label and the other three nucleotides with another label.

Binary sequencing may also be employed in which two bases, such as A and T (or U), are labeled with one label, while the other two bases, such as G and C, are labeled with a second label. A subsequent sequencing in which the label in one of each pair is changed, e.g., A and G are labeled with one label, and T (or U) and C are labeled with the other, may be employed to obtain base specificity.

One label may be employed, where the other three nucleotides are not labeled but are kept at lower concentrations, where the time, and therefore position, between the detection of each label is determined. Subsequent experiments using the same label with a different nucleotide can be used to provide the remaining sequence information.

When more than one labeled nucleotide is employed, each label may become light emitting as a result of different mechanisms. For example, one label may require additional chemical reaction after cleavage from the synthesized nucleic acid, while a second label may become light emitting upon cleavage without additional reaction.

Sequencing may also be performed using ligase, in which oligonucleotides hybridized adjacent to one another on a template strand are ligated together. Each oligonucleotide employed may be uniquely labeled. Oligonucleotides having the sequence complementary to a region of repeated sequence may be added sequentially using the methods of the invention, and the number of repeats determined by the number of oligonucleotides ligated.

Many proteins and enzymes require metallic co-factors such as divalent metal cations (Mg²⁺, Mn²⁺, Zn²⁺, etc.). For example, magnesium ions may be required for nucleic acid polymerase and alkaline phosphatase activity; manganese ions may be required to enhance the ability of the nucleic acid polymerase to incorporate modified nucleotide substrates (as described in U.S. Pat. No. 7,125,671 and Tabor S., Richardson C. C., Proc. Natl. Acad. Sci. USA, 1989, 86, 4076-4080); and zinc ions may be required for alkaline phosphatase activity. The presence of metal ions at high concentrations can complicate protein-protein interactions, protein-nucleic acid interactions, and surface passivation. In addition, divalent cations can destabilize polyphosphate compounds. Buffer components such as ammonium sulfate and chelating agents can be used to tune intermolecular interactions and control the effective concentration of metal ions. Many nucleic acid polymerizing replicating catalysts also require a reducing environment to perform optimally. There are many classes of reducing agents such as thiols (such as 2-mercaptoethanol or dithiothreitol) and phosphines (such as tris(2-carboxyethyl)phosphine (TCEP)), which are compatible with physiological buffers.

An individual sequencing reaction may be controlled by controlling the introduction of Mg or Mn ions, nucleotides, and other co-factors necessary to effect replication. Other methods for controlling replication include changing the temperature or introducing or removing substances that promote or discourage complex formation between the target and catalyst. The catalyst or target may also be rendered inoperative to end sequencing, e.g., through denaturation or cleavage.

Multiplexing, i.e., detection of more than one replication at a time, may also be employed to increase throughput.

### Fluorogenic Labels

Any label that becomes able to emit light as a result of incorporation of a nucleotide to a synthesized nucleic acid may be employed in the methods of the invention. Labels can be attached to nucleotides at a variety of locations. Attachment can be made either with or without a bridging linker to the nucleotide. The label may be attached to the base, sugar, or phosphate of the nucleotide. Preferably, the label is attached to the terminal phosphate, so it is cleaved from the nucleotide during replication. Labels may also be attached to non-naturally occurring portions of a nucleotide, e.g., to the delta or epsilon phosphate in a tetra- or pentaphosphate containing nucleotide. Alternatively, labels may be attached to the alpha phosphate and displaced during incorporation of a nucleotide in a synthesized strand.

In certain embodiments, the label is destroyed (or rendered non detectable) once detected. One method to destroy the label is photobleaching. Another method is to employ a catalyst that chemically alters the label after detection. In other embodiments, the label is not destroyed after detection, and the incorporation of nucleotides having the same label is monitored via the incremental increase of signal.

The properties of effective fluorophores for single molecule nucleic sequencing may differ substantially from than those required for bulk sequencing. Bulk nucleic acid sequencing reactions rely upon enzymatic amplification of nucleic acid molecules to generate large numbers of fluorescently labeled molecules for each sequenced base. The large numbers of labels detected relaxes constraints on the chemical stability, photostability, brightness, protein-dye interactions, as well as spectral separation between different labels. High fidelity single molecule sequencing typically requires greater constraints on these properties. Nucleic acid sequencing reactions also typically occur in a narrow range of conditions in which the polymerase and associated enzymes (such as alkaline phosphatase) operate optimally. These conditions vary considerably depending on the particular enzymes involved. One critical parameter with respect to fluorogenic label selection is the pH under which the sequencing reaction will take place (typically within the physiological pH range of 6 to 9), because the absorption and emission spectra of the product fluorophores are often strongly pH-dependent. For example, it is desirable for fluorogenic substrates that produce phenolic fluorophores to have pKₐ's below 7.

Below we list preferred criteria for fluorogenic labels for use in high-fidelity, single molecule fluorogenic sequencing:

1) No reactivity or detrimental interaction with buffer components, enzymes, nucleic acids, or other dyes or substrates.

Sequencing, and particularly single molecule sequencing, can involve a complicated set of proteins including nucleic acid polymerizing enzymes, enzymes to digest fluorogenic substrates resulting from the incorporation of labeled nucleotides (such as alkaline phosphatase), blocking proteins for surface passivation, and oxygen scavenger enzymes for mitigating photodamage. Nonspecific interactions between fluorogenic substrates/fluorophores with proteins can result in quenching via electron transfer, energy transfer, or chemical reactions that result in spectrally modified fluorophores. Such interactions can compromise nucleic acid sequencing by damaging the substrate, reducing fluorescence emission, or altering protein function. For example, many fluorophores have complicated interactions with reducing agents. In addition, proteins commonly have solvent exposed residues containing thiol moieties. The ground and excited states of several commonly used fluorogenic dyes such as resorufin and 7-hydroxy-9H-(1,3-dichloro-9,9-dimethylacridin-2-one) (DDAO) are susceptible to nucleophilic attack by thiols. Fluorescein analogs with certain patterns of halogenation are similarly vulnerable. Fluorogenic substrates may also be susceptible to nucleophilic attack by buffer components, despite the resistance of the corresponding fluorescent product. Fluorogenic substrates and fluorophores that react and interact minimally with the components of the sequence reaction are preferred for single molecule sequencing. Chemical modification can be rationally employed on the fluorogenic labels/fluorophores to impart resistance to these effects (see, e.g., U.S. Pat. Nos. 7,432,372, 6,162,931, and 6,229,055 and WO 2005/108994 A1).

2) Fluorogenic labels are preferably resistant to photodamage and preferably do not emit significantly in the detection band(s).

Because single molecule fluorogenic sequencing typically requires strong laser excitation of fluorescent label molecules, fluorogenic molecules within the detection volume are preferably substantially non-fluorescent when exposed to the excitation wavelengths. Preferably, these fluorogenic molecules have a very small extinction coefficient at these excitation wavelengths, such that they do not absorb photons when excited. Alternately, the fluorogenic molecules may have measurable absorbance at the excitation wavelengths of the fluorescent label, but thermal relaxation is the dominant process moving the substrate from the excited state to the ground state, substantially eliminating the possibility of fluorescence emission. In another embodiment, the substrate may absorb appreciably at the excitation wavelengths of the fluorescent label but emit fluorescence that is spectrally separated from the fluorescence generated by the fluorescent label. It is preferable for the fluorogenic substrate not to absorb the excitation light significantly, to limit time spent in the excited state, reducing the potential for any excited-state chemistry or bleaching.

3) Preferably, fluorophores produce a high photon flux at visible wavelengths with minimal blinking and bleach on a reasonable timescale.

Preferred fluorescent labels generate large photon fluxes (with high quantum efficiency) at wavelengths well-separated from the excitation wavelength and bleach in a single step yielding breakdown products that are substantially unreactive. This bleaching will occur on a reasonably short timescale, such that the generation of subsequent fluorescent labels can be detected in a background-free manner.

If the photon flux of the molecules is not constant (i.e., if the molecule “blinks”), these variations in fluorescence emission may be misinterpreted as the generation and subsequent bleaching of multiple fluorescent labels. Blinking can be caused by a variety of mechanisms, including visitation of a long-lived triplet state. A variety of methods for reducing the lifetime of this triplet state are described in US 2007/0161017 A1, which is hereby incorporated by reference.

The presence of molecular oxygen in the reaction chamber can also bleach fluorophores, reducing the average total number of photons generated. A variety of methods for eliminating molecular oxygen from a reaction sample (including enzymatic systems of catalase and glucose oxidase or protocatechuate 3,4-dioxygenase) are known in the art (see, e.g., US 2007/0161017 A1).

Alternately, molecular oxygen concentration can be used to control the average bleaching time of the fluorophore such that a detectable number of photons are emitted prior to bleaching, but only modest laser powers are necessary to bleach the fluorophore. Other molecules are also known to affect the photostability of different fluorophores, such as peroxide and reducing agents (such as DTT, TCEP, and BME). The time or excitation power required to eliminate the fluorophore once detection has occurred may be controlled using these compounds that have either excited-state or ground-state reactivity with the generated fluorophore.

Transient interactions with a surface (e.g. the surface of the microreactor) or buffer components, such as proteins at high concentration in the sequencing mixture, may quench fluorescence, creating spurious signal variations. Because high protein concentration in solution can cause nonspecific quenching of single molecules, an example of a protein-free system for reducing nonspecific adsorption to surfaces is also described herein.

Exemplary labels include resorufin and 9H-(1,3-dichloro-9,9-dimethylacridin-2-one) (DDAO). Additional labels are known in the art, e.g., in U.S. Pat. Nos. 7,041,812, 7,052,839, 7,125,671, 7,223,541, and 7,244,566.

Previous embodiments of fluorogenic nucleic acid sequencing have relied on a relatively narrow class of fluorogenic dyes for labeling nucleotide substrates (e.g., U.S. 2004/015119 and U.S. Pat. No. 7,125,671). In particular, phenolic dyes such as fluoresceins, phenoxazines (such as resorufin), acridines (such as DDAO), and coumarins may be used in fluorogenic substrates. The chemistry of fluorogenic nucleic acid substrates based on phenolic dyes is relatively straightforward because the phenolic oxygen is esterified to a phosphate group as in:

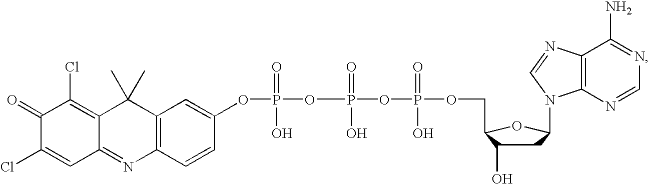

with the phenolic oxygen atom in bold. This substrate chemistry excludes the use of other potentially useful fluorogenic dyes such as those containing amines (e.g., rhodamine and its derivatives, cresyl violet, etc.).

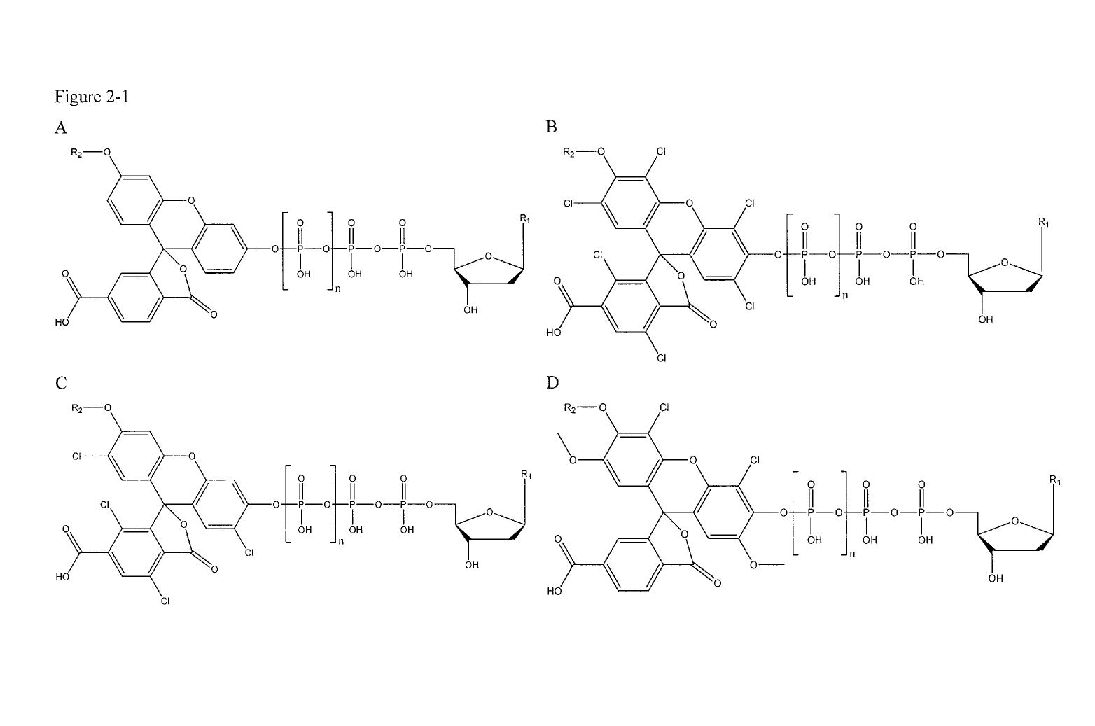

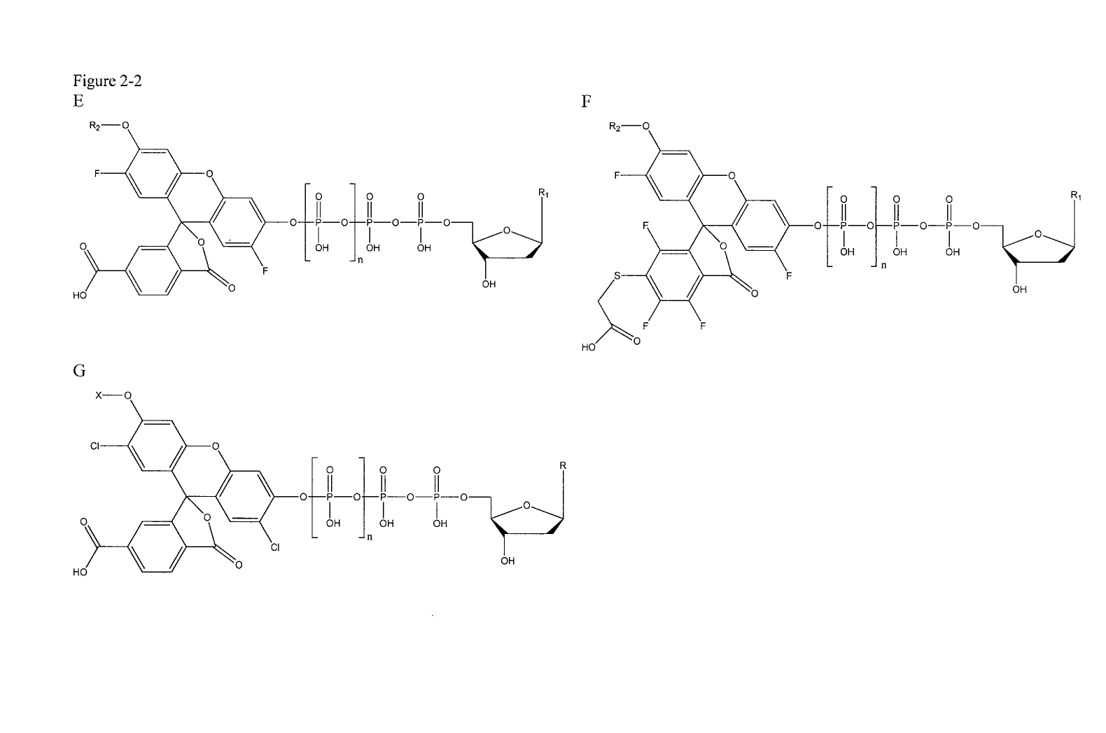

The invention also provides several classes of molecules for single-molecule sequencing. The first of these includes fluorogenic nucleotide substrates that employ a fluorescein-based fluorophore:

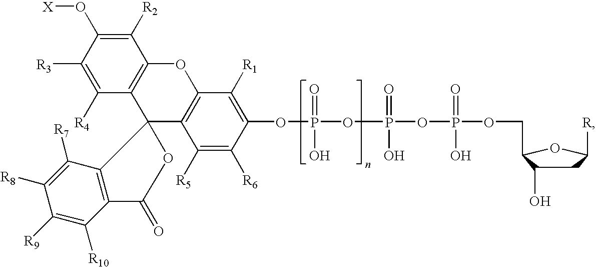

where R is a nucleoside base, as described herein, and X is a blocking group that serves to minimize the fluorescence emission of the substrate molecule. This blocking group is, for example, an alkyl group (e.g., such as methyl, ethyl, propyl, isopropyl, butyl), an acyl group (e.g., acetyl), sulfonyl (e.g., SO₂R, where R is C₁-C₆ alkyl), an alkyl group interrupted with one or more heteroatoms (e.g., O, N, S, or P), haloalkyl group (e.g., perfluorinated alkyl), cycloalkyl (e.g., with 3-6 ring carbons), carboxy substituted alkyl, sulfonyl substituted alkyl, or any other functional group that prevents the electronic structure of the attached oxygen from imparting significant fluorescence to the substrate molecule (see, e.g., WO 2005/108994). The functional groups R₁-R₁₀ are chosen to enhance the properties of the fluorogenic substrate and corresponding fluorophore to satisfy the requirements for single molecule nucleic acid sequencing described above. These groups may be selected from hydrogen, halogen (e.g., F), sulfonate (i.e., SO₃H), carboxy, acyl, alkyl, alkoxy, alkylthio, aryl, heteroaryl (e.g., containing one of O, N, or S), nitro, and hydroxyl (see also U.S. Pat. Nos. 7,432,372, 6,162,931, and 6,229,055 and WO 2005/108994 A1). Particular examples of fluorogenic nucleotide substrates with these modifications are shown in FIG. 2.

Another class of fluorogenic substrates have the general formula:

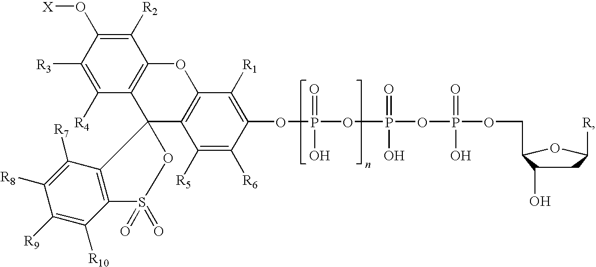

with R, X, and R₁-R₁₀ as described above. The fluorogenic dyes used in these substrates can be synthesized using methods known in the art (U.S. Pat. No. 6,130,101, U.S. 2005/0026235, and Pongev et al., Rus. J. Gen. Chem., 2001), and the corresponding substrates can be generated using the procedure described in Example 4.

A third class of fluorogenic compounds has the following structure:

Base-Sugar-Phosphate-[Self-reacting Component],

where Base is any nucleotide base as described herein, Sugar is any sugar or other such group in a nucleotide as described herein, Phosphate is a polyphosphate, and Self-reacting Component is a moiety that undergoes an intramolecular reaction upon cleavage of the phosphate to which it is connected to form a fluorophore. These compounds are substantially non-fluorescent at the wavelengths where the corresponding fluorophore emits and typically absorb very little at the absorption maximum of the corresponding fluorophore. The Self-reacting Component is of two forms. In one, this component includes a self-immolative linker conjugated to a fluorophore, wherein the conjugation renders the fluorophore substantially non-fluorescent. When the phosphate group is cleaved from the self-immolative linker, it spontaneously reacts, resulting in release of the fluorophore, which is fluorescent again. In another form, this component includes a proto-fluorophore, which is substantially nonfluorescent. Cleavage of the phosphate group from the proto-fluorophore results in an intramolecular reaction, e.g., lactonization, that forms a fluorophore. It will be understood that the compounds depicted above will be linked as is known in the art to produce a nucleotide, as defined herein, having a fluorogenic label.

An example of a fluorogenic substrate having a self-immolative linker is as follows:

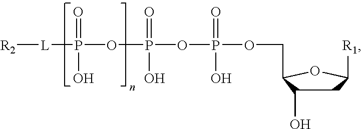

where R₁ is a nucleotide base, L is a self-immolative linker, n is an integer ranging from 0 to 4, and R₂ is a fluorogenic moiety.

Self-immolative linkers are known in the art (see, e.g., Zhou et al., ChemBioChem, 2008, 9, 714-718; Levine et al., Molecules, 2008, 13, 204-211; Lavis et al., ChemBioChem, 2006, 7, 1151-1154; Richard et al., Bioconjugate Chemistry, 2008, 19, 1707-1718; U.S. 2005/0147997; and U.S. 2006/0003383). An example of a self-immolative linker is the trimethyl lock linker (Levine et al., Molecules, 2008, 13, 204-211 and Lavis et al., ChemBioChem, 2006, 7, 1151-1154):

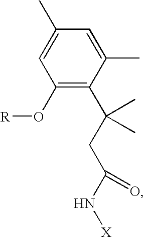

where R is an enzyme substrate moiety (e.g., phosphate), and X—NH₂ is a fluorophore. A fluorogenic nucleotide substrate having the trimethyl lock has the general structure:

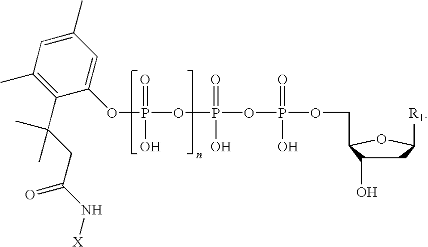

One class of amine-containing fluorophores includes rhodamine derivatives, where the corresponding nucleotide substrate has the general structure:

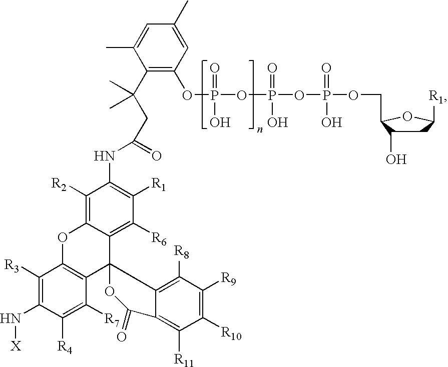

where R is a nucleotide base, n is an integer ranging from 0 to 4, and X is a blocking group (as discussed above) that serves to minimize the fluorescence emission of the chromophore when it is conjugated to the substrate. The groups R₁-R₄ and R₆-R₁₁ are all hydrogen atoms in the case of rhodamine but can be modified to form derivatives with different chemical, spectral, and photophysical properties. R₁-R₄ and R₆-R₁₁ can be hydrogen, halogen (e.g., F), sulfonate, carboxy, acyl, alkyl, alkoxy, alkylthio, aryl, heteroaryl (e.g., containing one of O, N, or S), nitro, or hydroxyl, which may be substituted as described herein. Exemplary rhodamine dyes include rhodamine B, rhodamine 19, rhodamine 110, rhodamine 116, sulforhodamine B, and carboxyrhodamine.

Derivatives of oxazine dyes can also be employed in a similar fashion:

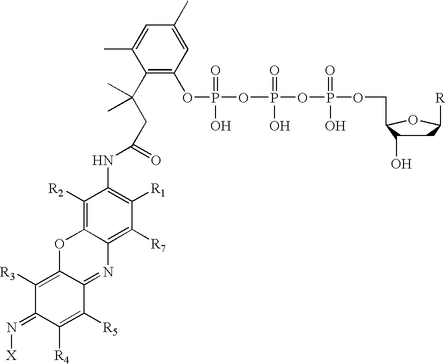

where R is a nucleoside base, n is an integer between 0 and 4, X is a blocking group (as discussed above) that serves to minimize the fluorescence emission of the chromophore when it is conjugated to the substrate, and R₁-R₅ and R₇ represent functional groups as discussed for rhodamine. An exemplary oxazine dye is 3-imino-3H-phenoxazin-7-amine (oxazine).

Benzophenoxazine dyes, such as cresyl violet and its derivates, can also be employed:

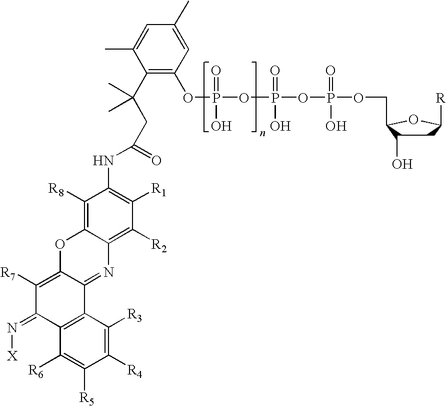

where R is a nucleoside base, n is an integer between 0 and 4, X is a blocking group (as discussed above) that serves to minimize the fluorescence emission of the chromophore when it is conjugated to the substrate, and R₁-R₈ represent the functional groups as discussed for rhodamine. An example of a benzophenoxazine dye is 9-imino-9H-benzo[a]phenoxazine-5-amine.

These compounds will be incorporated by a nucleic acid replicating catalyst into a nucleic acid and yield a polyphosphate chain terminated by the self-immolative linker conjugated to the fluorophore:

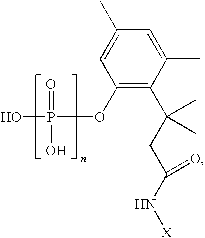

where X—NH₂ is a fluorophore. A phosphatase can then be used to cleave the polyphosphate chain leading to the generation of the following species:

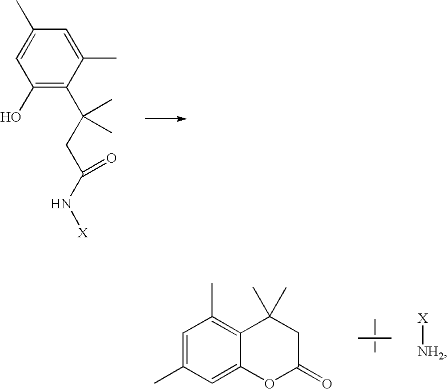

resulting in the generation of an amine-containing fluorophore, which can be detected at the single molecule level.

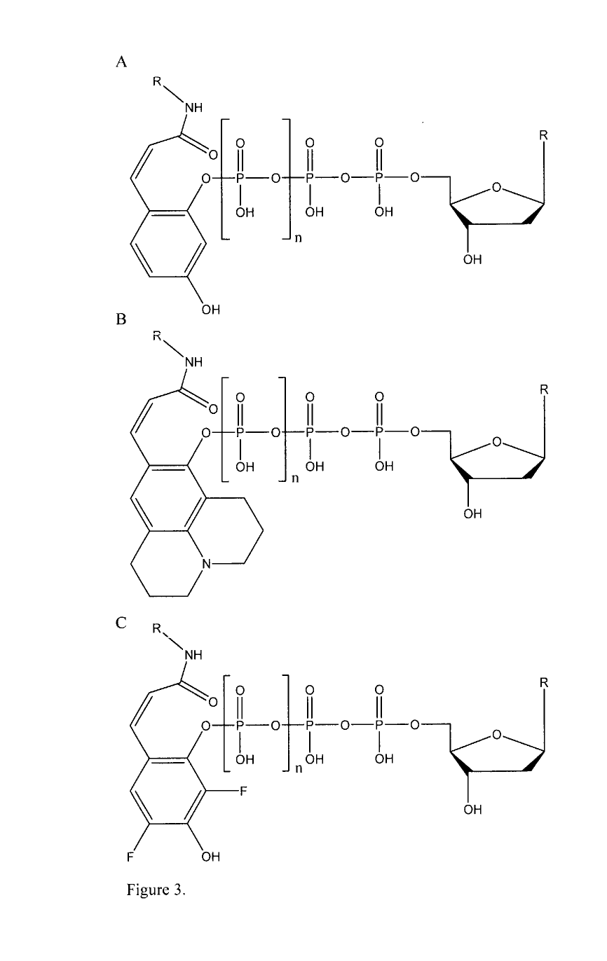

The Self-reacting Component may also result in spontaneous generation of a fluorophore, e.g., through cyclization reactions in response to enzymatic digestion. Fluorogenic nucleotide substrates based on self-generating fluorophores with the general structure given below can be used for nucleic acid sequencing:

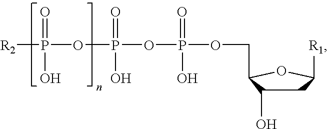

where R₁ is a nucleotide base, n is an integer between 0 and 4, and R₂ is a moiety that undergoes an intramolecular reaction to form a fluorophore upon removal of the phosphate. An example of these compounds results in generation of a coumarin fluorophore (see, e.g., Wang et al., Methods in Molecular Medicine, 1998, 23, 71; Wang et al., Bioorganic and Medicinal Chemistry Letters, 1996, 6, 945-950; and U.S. Pat. No. 6,214,330):

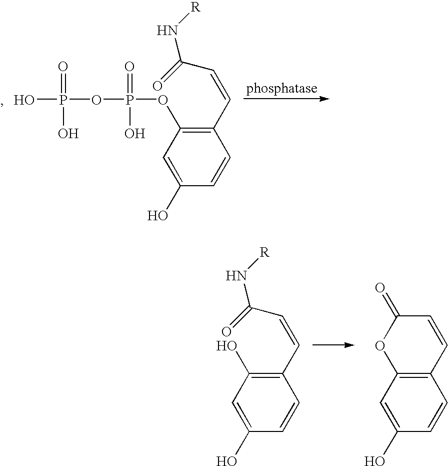

where R represents any suitable substituent for the amine leaving group. FIG. 3 shows examples of these nucleotide substrates.

It will also be understood that the sugar moiety depicted in any of the above structures, i.e., 2′-deoxyribose, may be replaced with any other appropriate group, as described herein (for example, the nucleotide may be a ribonucleotide).

### Microreactors

The reagents for synthesis of nucleic acids are disposed in a microreactor. Exemplary microreactors hold volumes of 0.0001 fL to 1000 fL, although larger volumes are possible. Conducting single molecule sequencing in a microreactor imparts several advantages as described herein.

A target nucleic acid, activating catalyst, or replicating catalyst may be immobilized within the microreactor, although the methods of the invention do not require immobilization. Methods for immobilizing nucleic acids or catalysts are well known in the art and include biotin-streptavidin, antibody-antigen interactions, covalent attachment, or attachment to complementary nucleic acid sequences. In certain embodiments, it is preferred for the nucleic acid to be immobilized, e.g., on a bead or to the microreactor, to allow for repeated sequencing (or synthesis of copies).

A target nucleic acid or, activating catalyst, or replicating catalyst may be immobilized to beads (magnetic, paramagnetic, polystyrene, glass, etc.) using immobilization techniques well known in the art. When the nucleic acid is immobilized to a bead, these beads can then be trapped in microreactors, and the nucleic acid can be directly sequenced with our method. Affinity capture beads may also be used to capture relevant nucleic acids, e.g. eukaryotic RNA can be specifically extracted by annealing poly-dT coated beads to the poly-A tail of the mRNAs.

Materials that are useful in forming the microreactors include glass, glass with surface modifications, silicon, metals, semiconductors, high refractive index dielectrics, crystals, gels, lipids, and polymers (e.g., poly(dimethyl siloxane) (PDMS)). Mixtures of materials may also be employed.

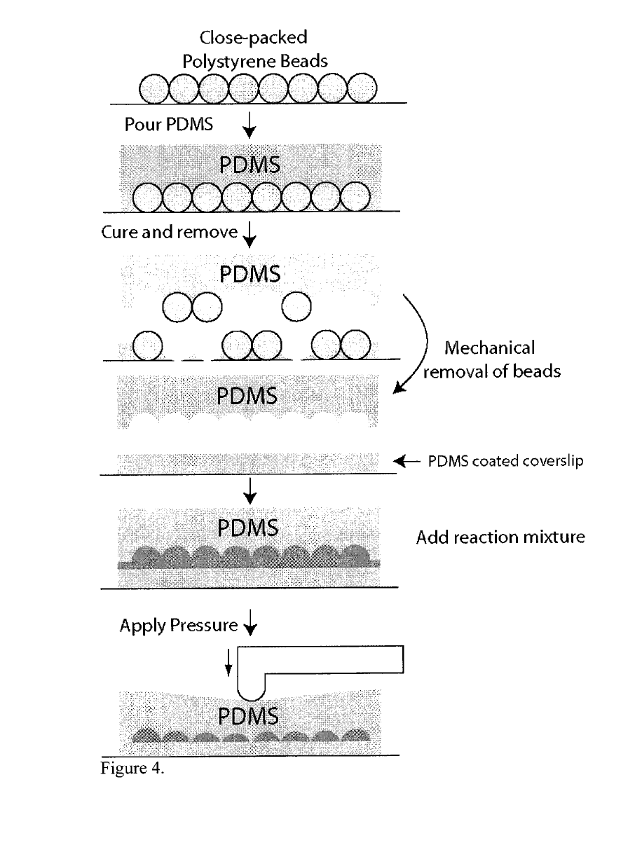

An exemplary method of fabricating microreactors in PDMS is described herein (FIG. 4). Other materials for microreactor fabrication include polytetrafluoroethylene, perfluoropolyethers, and parylene. Additionally, lipid vesicles can be generated using standard lipid extrusion techniques (Okumus et al. Biophys. J. 2004, 87(4), 2798-2806) and used to confine the reaction. Another method of generating microreactors is the creation of an emulsion of the reaction mixture in an immiscible solvent such as mineral oil or silicon oil. These and other methods for manufacturing microreactors are known in the art, e.g., U.S. Pat. Nos. 7,081,269, 6,225,109, 6,225,109, and 6,585,939.

Single molecules of target nucleic acid (or replicating catalyst) can be delivered to a microreactor using methods known in the art. One method for delivery is to provide a dilute solution of nucleic acid so that each microreactor, on average, holds less than one molecule. Using this approach some microreactors will have no target nucleic acid, some will have a single target nucleic acid, and a very small number will have more than one. The same strategy may be employed to attach a single molecule of nucleic acid to a bead or to the lid of a microreactor.

Fluorophores and fluorogenic labels are preferably trapped in the microreactor during the course of a sequencing. If either the generated fluorophore or the fluorogenic-label escapes the reactor, then information regarding the sequencing of the nucleic acid may be lost. Materials and methods for retaining fluorophores and fluorogenic substrates within a reactor are described herein.

Microreactors are preferably manufactured from materials that prevent or reduce diffusion of fluorophores, evaporation of water, and nonspecific absorption of proteins. Alternatively, microreactors are treated to prevent or reduce such diffusion, evaporation, and nonspecific absorption. Treatment methods are described herein.

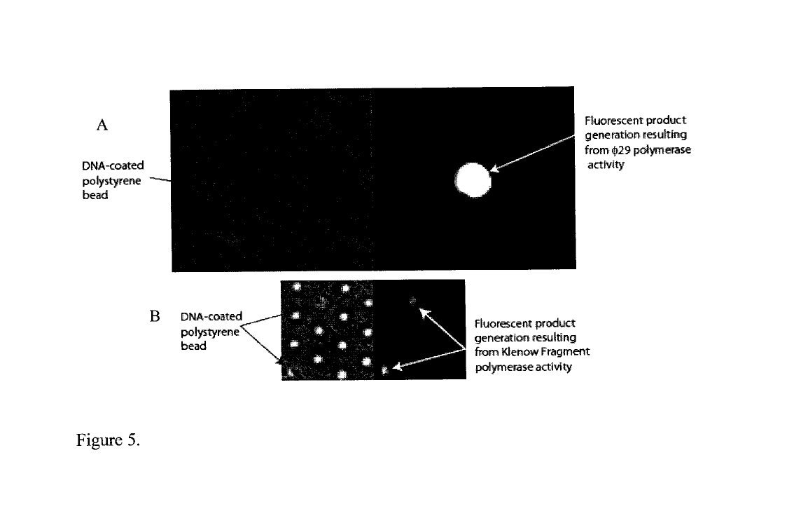

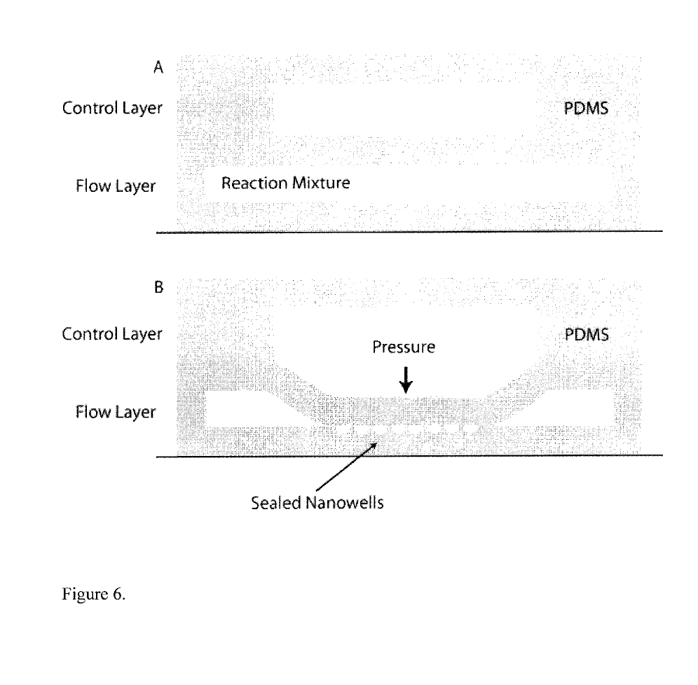

Microreactors may or may not have lids to enclose the reaction mixture. When a lid is employed, the nucleic acid may be immobilized on it. The lid can be sealed by conformal pressure, adhesives, and other bonding techniques known in the art. An exemplary process for sealing microreactors made from PDMS (or other elastomeric materials) is shown in FIG. 6. This process employs valve technology known in the art (Unger, M. A. et al. 2000. Science, 288, 113-116; Jung et al. Langmuir, 2008. 24, 4439-4442). Lids made from glass and other optical quality materials are preferred.

### Activating Catalyst

Any catalyst that is capable of acting on a label to render it fluorescent after a nucleotide incorporation event may be used in the invention. Preferably, the activating catalyst does not act on the label prior to incorporation. Preferred catalysts include enzymes such as alkaline phosphatases (e.g., bacterial alkaline phosphatase, shrimp alkaline phosphatase, calf intestinal phosphatase, and antarctic phosphatase), acid phosphatases, galactosidases, horseradish peroxidase, phosphodiesterase, phosphotriesterase, pyruvate kinase, lactic dehydrogenase, lipase, or combinations of enzymes and substrates in a coupled enzyme system such as maltose, maltose phosphorylase, glucose oxidase, horseradish peroxidase, and amplex red (PIPER™ phosphate detection kit, Invitrogen). The activating catalyst may also be an ion in solution, e.g., iodide, hydroxide, or hydronium, a zeolite or other porous catalytic surface, or a metal surface, e.g., platinum, palladium, or molybdenate. Other biological and synthetic catalysts may also be employed. Multiple copies of a particular catalyst may be present to reduce the time required for interaction with the label. The catalyst may be immobilized to a surface of the microreactor or a bead to increase the effective concentration within the reactor.

### Nucleic Acids and Nucleotides

The invention may be employed with any nucleic acid (e.g., DNA, RNA, and DNA/RNA) using any appropriate nucleic acid replicating catalyst. Nucleotides may be naturally occurring or synthetic, e.g., synthetic ribonucleosidyl, 2′-deoxyribonucleosidyl, Locked Nucleic Acid, peptide nucleic acid, glycerol nucleic acid, morpholino nucleic acid, or threose nucleic acid connected, e.g., via the 5′, 3′, or 2′ carbon of the radical, to a phosphate group and a base. The nucleotide may include a purine or pyrimidine base, e.g., cytosine, guanine, adenine, thymine, uracil, xanthine, hypoxanthine, inosine, orotate, thioinosine, thiouracil, pseudouracil, 5,6-dihydrouracil, and 5-bromouracil. The purine or pyrimidine may be substituted as is known in the art, e.g., with halogen (i.e., fluoro, bromo, chloro, or iodo), alkyl (e.g., methyl, ethyl, or propyl), acyl (e.g., acetyl), or amine or hydroxyl protecting groups. In certain embodiments, the nucleotides employed are dATP, dCTP, dGTP, and dTTP. In other embodiments, the nucleotides employed are ATP, CTP, GTP, and UTP. Ribosides may be employed for sequencing DNA, e.g., when DNA-dependent RNA polymerase is employed. Ribosides may be employed for sequencing RNA, e.g., when RNA-dependent RNA polymerase is employed. Deoxyribosides may also be employed for sequencing RNA, e.g., when reverse transcriptase is employed. In preferred embodiments, the sequencing methods of the invention produce a nucleic acid that is complementary to the target nucleic acid and that includes only naturally occurring nucleotides, i.e., the label is removed during incorporation. Alternatively, nucleotides may include a moiety that is retained in the synthesized nucleic acid. Such moieties are preferably present on fewer than all of the labeled nucleotides employed, e.g., only one, two, or three, to minimize disruption of replicating catalyst activity.

### Nucleic Acid Replicating Catalysts

Exemplary replicating catalysts include DNA polymerases, RNA polymerases, reverse transcriptases, ligases, and RNA-dependent RNA polymerases. Exemplary DNA polymerases include E. coli DNA polymerase I, E. coli DNA polymerase I Large Fragment (Klenow fragment), Klenow fragment (exo-), Sequenase™, phage T7 DNA polymerase, T4 DNA polymerase, Phi-29 DNA polymerase, Phi-29 (exo-) DNA polymerase, Bsu DNA polymerase (exo-), thermophilic polymerases (e.g., Thermus aquaticus (Taq) DNA polymerase, Thermus flavus (Tfl) DNA polymerase, Thermus thermophilus (Tth) DNA polymerase, Thermococcus litoralis (Tli) DNA polymerase, Pyrococcus furiosus (Pfu) DNA polymerase, Vent™ DNA polymerase, or Bacillus stearothermophilus (Bst) DNA polymerase, Therminator™, Therminator II™, Therminator III™, and Therminator-γ™), and reverse transcriptase (e.g., AMV reverse transcriptase, MMLV reverse transcriptase, SuperScript-1™, SuperScript-2™, SuperScript-3™, or HIV-1 reverse transcriptase). In addition, existing polymerase enzymes can be rationally mutated or selected using directed evolution to enhance the efficiency and fidelity with which they incorporate modified nucleotides (U.S. 2007/0196846, U.S. 2007/0172861, and U.S. 2007/0048748). Other suitable DNA polymerases are known in the art. Exemplary RNA polymerases include T7 RNA polymerase, T3 RNA polymerase, SP6 RNA polymerase, and E. coli RNA polymerases. Exemplary ligases are known in the art. Exemplary RNA-dependent RNA polymerases are known in the art. Catalysts may bind to a target at any appropriate site as is known in the art.

Multiple copies of the replicating catalyst may be present. If a particular catalyst molecule disassociates from the template strand, another catalyst molecule may bind and continue replication without affecting the sequencing function.

### Detection

Incorporation of an individual nucleotide may be detected by detecting the light emitted from its corresponding label by any appropriate method. For fluorescent labels, one or more excitation sources may be employed, depending on the nature and number of labels. Methods for single molecule detection are known in the art. Examples are conventional fluorescence microscopy, total internal reflection fluorescence microscopy, or parallel confocal microscopy (Lundquist et al. Optics Letters. 2008 33(9) 1026-1028). As described above, the methods of the invention may be employed in a multiplexed mode, where the sequences of multiple target nucleic acids are determined simultaneously, e.g., using a wide field of view detector such as a charge-coupled device (CCD) or multiple detectors.

### Microfluidic Sample Preparation

In certain embodiments, target nucleic acids are purified from crude biomaterials (such as blood, tissue, etc.) using microfluidic techniques, which may be integrated with a system of the invention. Methods for isolating nucleic acids from cellular samples using microfluidic devices (i.e., devices having a channel with at least one dimension of less than 1 mm) are known in the art (e.g., U.S. Pat. No. 6,352,838). In addition, microfluidic devices may also be used to obtain either RNA or DNA from a single cell, e.g., as described Toriello et al., Proc. Natl. Acad. Sci., 2008 105(51), 20173-20178.

### Example 1

In this example, 500 nm streptavidin-coated polystyrene beads (Bangs Laboratories) were incubated at a concentration of 50 μM for 20 minutes in reaction buffer (50 mM Tris-HCl pH 8, 50 mM NaCl, 0.1% Tween-20, 0.2% Pluronic-F108, 1% PEG-10K) with 5 nM biotinylated template DNA (a primed poly-C homopolymer) on ice. The composition of the reaction mixture was then adjusted to include dGTP-γ-resorufin (20 μM), MnCl₂ (1 mM), SAP (1 μM), and either φ29 (exo-) DNA polymerase or Klenow fragment (exo-) DNA polymerase on ice. When Klenow fragment (exo-) DNA polymerase was used, 0.25 mM DTT was included in the reaction mixture. The reaction mixture was immediately sealed in PDMS microreactors (either 5 μm or 1.5 μM in diameter) and imaged on a fluorescence microscope.

A microscope (Nikon TE-2000 with 60×1.2 NA water-immersion objective) was operated in wide-field fluorescence mode with 560 nm laser excitation. Bright field and fluorescence signals were imaged onto an EM-CCD camera (Cascade 512B, Roper Scientific). The resulting images are shown in FIG. 5.

### Example 2

In this example, deoxynucleotide triphosphates (dNTPs) derivatives that are linked through the γ-phosphate to different dyes, which are essentially non-fluorescent at relevant wavelengths in solution, are synthesized. High concentrations of labeled dNTPs may thus be present in solution without fluorescence background, as these molecules are “dark.” Once a DNA polymerase incorporates a labeled dNTP, cleaving between the α- and β-phosphates of the nucleotide, the liberated fluorophore becomes fluorescent, either directly upon cleavage from the dNTP, or after further enzymatic action of other enzymes (Sood et al. J. Am. Chem. Soc., 2005, 127, 2394-2395 and Kumar et al. Nucleotides, Nucleosides, and Nucleic Acids, 2005, 24, 401-408) (through a coupled enzyme assay discussed further below). These newly fluorescent molecules are then detected using standard fluorescence detection techniques (English et al. Nat. Chem. Biol., 2006, 2, 87-946) (such as total internal reflection fluorescence, epifluorescence, or confocal microscopy). The color of the fluorescence reports the identity of the incorporated nucleotide, and thus the underlying DNA sequence.

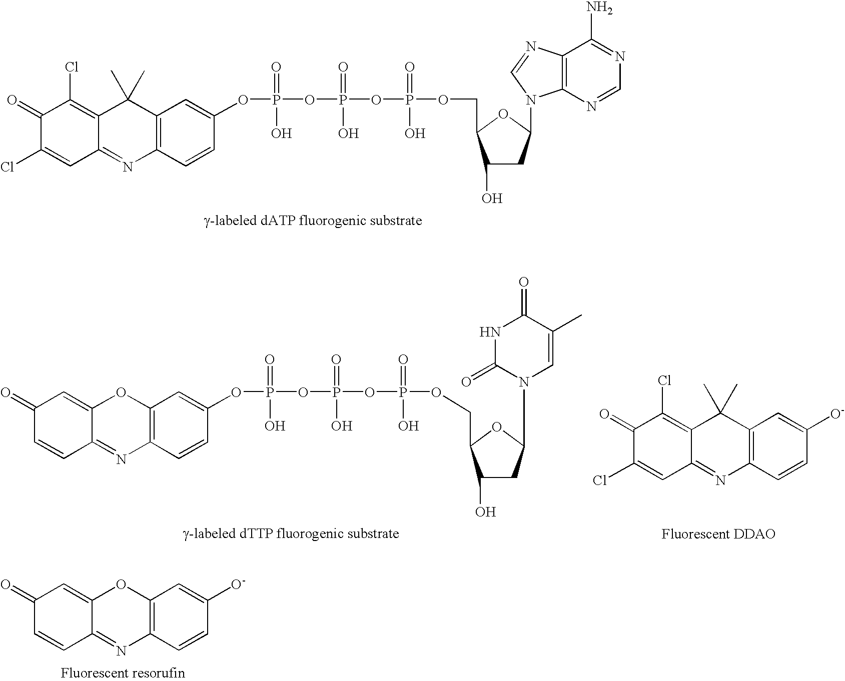

In one preferred implementation of the invention, we use two different fluorogenic species (DDAO and resorufin, above) to extract sequence information from DNA. Resorufin is not fluorescent when conjugated to dNTPs, while for DDAO the fluorescence and absorption spectra change significantly when it is conjugated to dNTPs (see above). Upon cleavage from the dNTP through the action of DNA polymerase, these molecules still have phosphate groups covalently linked to the fluorophore, which must be removed before the molecule may become fluorescent. A large class of phosphatase enzymes, such as shrimp alkaline phosphatase, can quickly remove these phosphate groups from the fluorophore (but do not react with γ-labeled dNTP substrates), generating a single fluorescent dye molecule for every incorporation of the DNA polymerase (see FIG. 1). However, to use this coupled-enzyme assay, the biochemical reaction must be conducted in a confined volume such that the freed, phosphate-attached fluorophores do not diffuse away from the site of the DNA incorporation.

One method to achieve this confinement is the use of sub-micron lipid vesicles to entrap DNA, substrate, DNA polymerase, and phosphatase. We then immobilize these “microreactors” on the coverslide of a fluorescence microscope (Okumus et al. Biophys. J. 2004, 87(4), 2798-2806). An alternate approach utilizes standard nanofabrication techniques to generate femtoliter-sized indentations in PDMS, poly(methyl methacrylate) (PMMA), or quartz, which we can seal against the surface of a coverslide (Rondelez et al. Nature Biotechnology. 2005, 23, 361-5 and Jung et al. Langmuir. 2008, 24, 4439-4442).

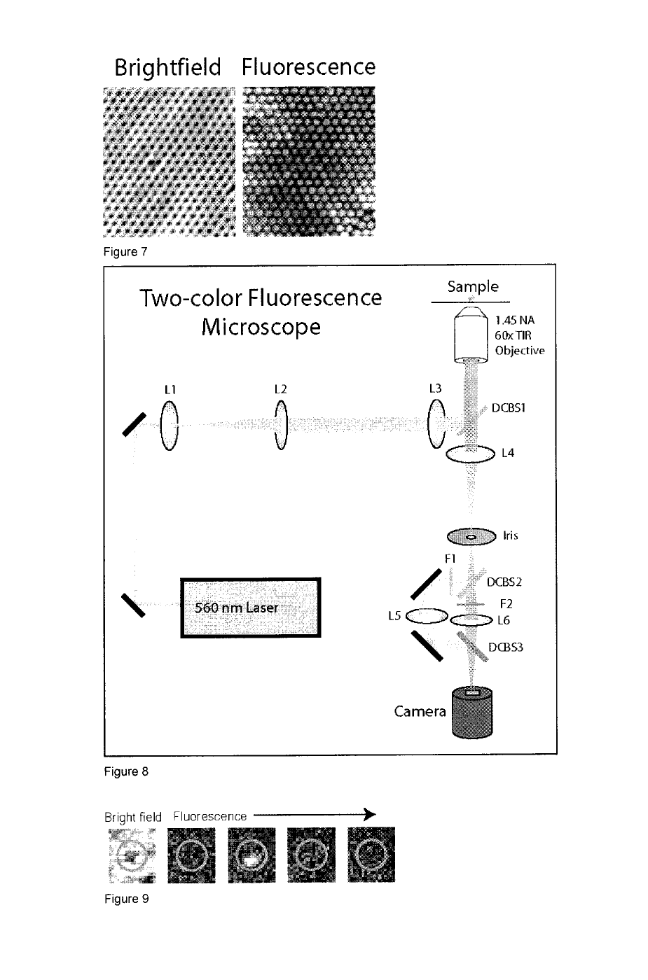

These microreactors may also be generated through a variant of so-called nanosphere lithography (Hulteen et al. J. Vac. Sci. Technol. A 1995 13(3), 1553-1558) (see FIG. 4). In brief, we evaporate 500 nm to 2000 nm polystyrene or glass beads on glass slides to create a close-packed monolayer of beads. Then we pour PDMS onto these close-packed regions and cure the PDMS in a 60° C. oven overnight. The cured PDMS can then be peeled away from the glass, and impregnated beads removed mechanically. This process produces a portion of PDMS with a pattern of nanoscale indentations reminiscent of a honeycomb. Then, this PDMS pattern of dimples was pressed against a PDMS spin-coated coverslip to generate a regular array of microreactors that contain on the order of 5 to 0.1 fL. We are able to trap dye in these microreactors (see FIG. 7) and image the dye with a two color TIRF microscope (see FIG. 8).

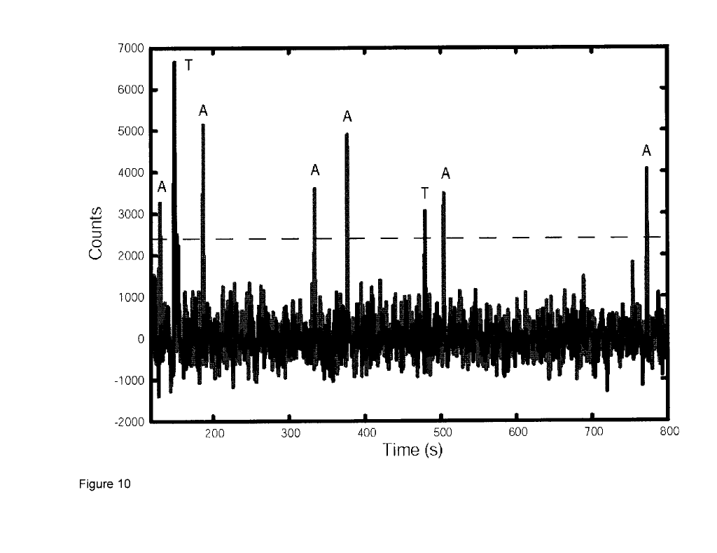

In these microreactors, individual molecules of DNA were synthesized from a single-stranded template, generating a fluorescent molecule (through the combined action of the polymerase and phosphatase) for every incorporated base. With a TIRF microscope (FIG. 8), we detected the generation of these fluorescent molecules, and bleached them (see FIG. 9). We detected single molecular fluorescence flashes created by nucleotide incorporation coupled with phosphatase activity (FIG. 10). In this implementation, resorufin was generated for incorporations of dTTP, and DDAO was generated for incorporations of dATP.

### Example 3

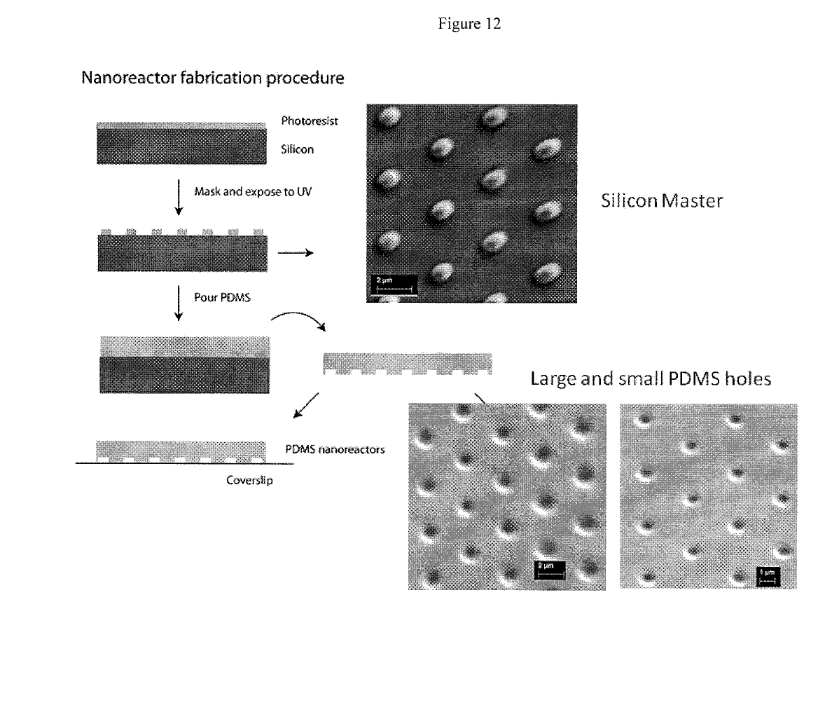

We fabricated microreactors to trap fluorophores and fluorogenic substrates. To improve the sealing characteristics of PDMS microreactors, we used standard photolithographic methods to construct a microreactor array with wall thickness of greater than 1 micron. First, a flat 3 inch silicon wafer was coated with 0.5-1.5 microns of SU-8 2 photoresist and prebaked for 60 seconds at 65° C. and then 60 seconds at 95° C. Next, this photoresist was exposed through a patterned, chrome-on-glass photomask to UV light, which cross links the photoresist. This wafer is then post baked (identically to the prebake step) and developed, resulting in a resist-on-silicon master (FIG. 12). Finally, PDMS was poured onto this master, cured, and then used in experiments (FIG. 12). We have created ˜0.5, ˜1, ˜1.5, ˜2, ˜5, and ˜20 micron diameter reaction chambers using these methods.

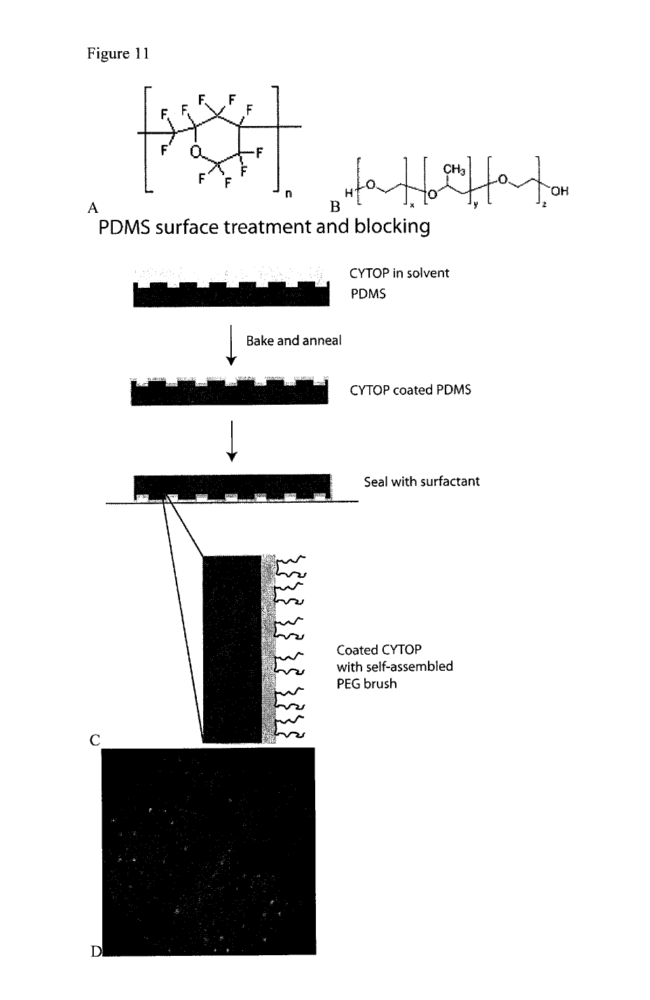

To reduce nonspecific absorption of proteins and other species, PDMS was coated with an amorphous fluoropolymer CYTOP (perfluoro(1-butenyl vinyl ether) homocyclopolymer from Asahi Glass Co., see FIG. 11A) by spincoating and baking at 75° C. for 15 minutes and 145° C. for 15 min (FIG. 11C). Then the CYTOP was coated with Pluronic F-108 (in the reaction solution), which spontaneously forms a polyethylene glycol brush on the surface of the microreactor because of hydrophobic interactions of the poly(propylene glycol) portion of the copolymer (FIG. 11B). We observed that this surface treatment prevents the adsorption of single fluorescently labeled protein molecules (FIG. 11D), thus eliminating the need for high concentrations of blocking protein (such as BSA). The treatment also renders PDMS hydrophilic. Alternatively, moderate concentrations of BSA (1 mg/mL) can be used to block the PDMS.

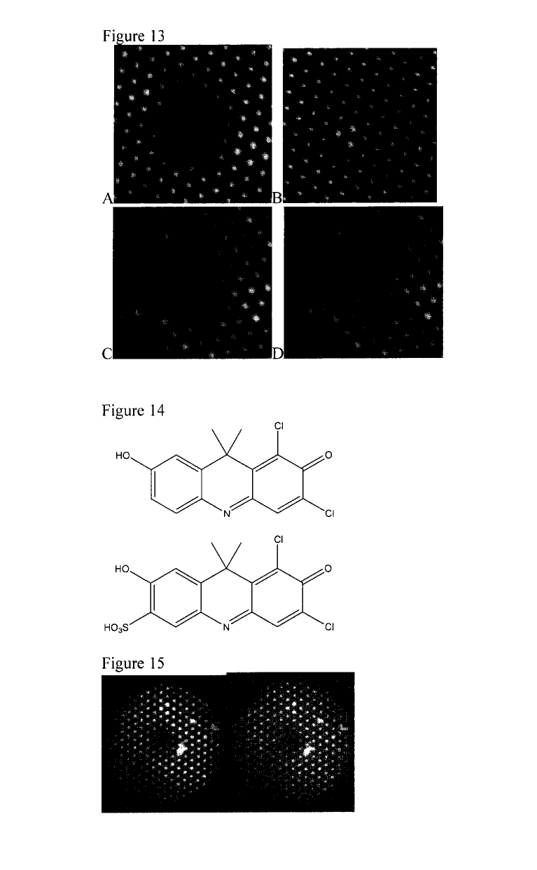

Dyes such as DDAO and resorufin may diffuse through PDMS microreactors, escaping the reactors in a timescale of seconds to minutes (FIG. 13-D). Dyes with local negative charge may be efficiently trapped in PDMS microreactors for long timescales, e.g., on the order of hours (see, e.g., Rondelez, Y. et al. Nat Biotech 23, 361-365(2005)). We demonstrated that the addition of a sulfonate group to DDAO (FIG. 14) provides the dye molecule with a local negative charge and eliminated diffusion of this dye through PDMS (FIG. 15). This finding confirms that dyes with local negative charge were trapped in the PDMS microreactors.

We also treated PDMS microreactors with a stable fluorocarbon fluid (such as Fluorinert FC-43 and FC-770, 3M). By treating the PDMS with these compounds, we reduced the incidence of evaporation of the liquid phase within the reaction chambers and also reduced diffusion of uncharged substrates within the PDMS.

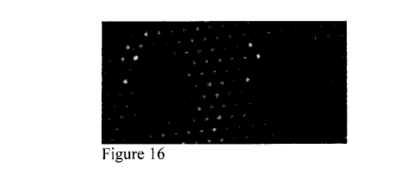

Alternately, microreactors are constructed out of different materials, such as fluorothermoplastics like THV 220 (3M), or PDMS can be coated with other impermeable materials to block the diffusion of non-charged dye species. Material coatings such as CYTOP also reduced or eliminated the diffusion of even non-charged dye molecules (FIG. 16). Additionally, coating a CYTOP layer with a fluorocarbon liquid (such as Fluorinert FC-43, 3M) allows more robust sealing of microreactors by filling in small imperfections in the CYTOP layer.

In addition, vapor phase treatment of the oxidized coverglass surface with a variety of reactive silanes such as 1H,1H,2H,2H-perfluorooctyltrichlorosilane or [tris(trimethylsiloxy)silylethyl]dimethylchlorosilane produces a hydrophobic surface that facilitates the robust sealing of PDMS microreactors. Also, this hydrophobic and/or fluorinated surface can be passivated effectively with nonionic detergents. Finally treatment of the surface with bi-functional reactive silanes, such as 3-mercaptopropyltrimethoxysilane (Liu et al. Langmuir, 2004, 20(14), 5905-5910), allows for direct, covalent coupling of protein, DNA, or other molecules such as biotin to the glass surface.

### Example 4

### General Synthesis Procedure for Fluorescein-Based Fluorogenic Nucleotide Substrates (see, e.g., WO 2005/108994)

Ethylation of a Fluorescein Derivative

A fluorescein analog having a carboxylic acid group at the six position was dissolved in methanol and combined with a three-fold molar excess of NaOH. The solution was evaporated, and the resulting solid was co-evaporated with anhydrous DMF three times. The solid was taken up in DMF at ˜40-50 mg/mL, and an 8-fold molar excess of iodoethane was added. The solution was stirred at room temperature for 8 hours, evaporated to dryness, and taken up in 50 mM sodium bicarbonate buffer (pH 8.5). The ethylated fluorescein derivative was purified via HPLC (XTerra C18 reverse phase column). At this point, the desired phenolic hydroxyl group has been converted to an ethyl ether, and any carboxyl groups have been converted to ethyl esters. Following purification, the ethylated product was dried and re-dissolved in 6N hydrochloric acid to hydrolyze any ethylated carboxyl groups to carboxylic acids. The solution was heated gently for 3 hours. The product was extracted with methylene chloride, evaporated to dryness, re-dissolved in 50 mM sodium bicarbonate buffer (pH 8.5), and re-purified via HPLC (XTerra C18 reverse phase column). This resulted in the 3-O-ethyl derivative of the particular fluorescein analog.

Phosphorylation of the Ethylated Fluorescein Derivative

The 3-O-ethyl derivative was dissolved to ˜40-50 mg/mL in anhydrous acetonitrile with a three-fold molar excess of Proton Sponge (Sigma). The mixture was co-evaporated with anhydrous acetonitrile three times before being re-dissolved in anhydrous acetonitrile under argon. The resulting solution was cooled to −5° C. A three-fold molar excess of phosphorus oxychloride was added, and the solution was stirred under argon for 90 minutes at −5° C. The reaction was quenched by the addition of 50 mM sodium bicarbonate buffer (pH 8.5). The resulting solution was evaporated to dryness, and the monophosphorylated 3-O-ethyl derivative of the fluorescein analog was purified via HPLC (XTerra C18 reverse phase column).

Synthesis of an Imidazole Derivative of the Ethylated, Phosphorylated Fluorescein Analog

The monophosphorylated, ethylated fluorescein analog with a three-fold molar excess of triethylamine were co-evaporated in anhydrous DMF three times. The resulting solid was re-dissolved in anhydrous DMF under argon, and a five-fold molar excess of carbonyldiimidazole was added. The mixture was stirred for 3 hours. About 0.4 μL methanol per 1.0 mg of carbonyldiimidazole was added to the mixture to quench the remaining carbonyldiimidazole. The solution was stirred for 30 minutes before being evaporated to dryness and re-dissolved in anhydrous DMF under argon.

Conjugation of the Imidazole Derivative of the Ethylated, Phosphorylated Fluorescein Analog to a Nucleotide Polyphosphate

The desired nucleotide polyphosphate (tributylammonium salt) was co-evaporated with anhydrous DMF three times. The resulting solid was re-dissolved in anhydrous DMF under argon. The DMF solution containing the imidazole derivative of the ethylated, phosphorylated fluorescein analog was added to the nucleotide polyphosphate (which is in a three-fold molar excess). The resulting solution was stirred at room temperature for two days before being concentrated to dryness, re-suspended in 50 mM triethylammonium bicarbonate buffer (pH 8.5), and purified via reverse phase HPLC to obtain a fluorogenic nucleotide polyphosphate substrate.

Generalized Synthesis of a Rhodamine-Based Fluorogenic Nucleotide Substrate Using the Trimethyl Lock
Synthesis of a Trimethyl Lock Phosphate (see, e.g., Zhou et al., ChemBioChem, 2008, 9, 714-718)

2-(3-hydroxy-1,1-dimethyl-propyl)-3,5-dimethyl-phenol is reacted with tert-butyl-dimethylsilyl chloride in methylene chloride. The resulting protected compound is purified with reverse phase HPLC. The protected compound is dissolved in methylene chloride with potassium tert-butoxide to which diethyl chlorophosphate is added to phosphorylate the protected compound at the phenolic oxygen. The still protected oxygen is converted to a carboxylic acid following deprotection with potassium fluoride by dissolving the product in acetone and reacting it with Jones' reagent at room temperature. The resulting product is evaporated to dryness, re-dissolved in 50 mM triethylammonium bicarbonate buffer (pH 8.5), and purified with reverse phase HPLC (XTerra C18 column) to obtain a pure sample of Compound A:

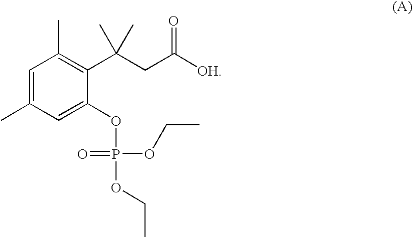

Synthesis of a Partially Blocked Rhodamine Analog (see, e.g., U.S. 2005/0147997)

Rhodamine 110 is dissolved in anhydrous DMF, and a 3-fold molar excess of N,N-diisopropylethylamine is added. The solution is stirred at room temperature for 5 minutes followed by the slow addition of 4-morpholinecarbonyl chloride (with the rhodamine analog in a 10-fold molar excess). The resulting solution is stirred at room temperature for two days before being evaporated to dryness and purified with reverse phase HPLC. This procedure results in Compound B:

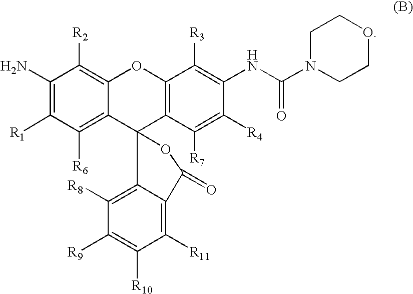

Conjugation of the Phosphorylated Trimethyl Lock to the Blocked Rhodamine
Derivative (see, e.g., Zhou et al., ChemBioChem, 2008, 9, 714-718)

Compound A is combined with isobutylchloroformate and Compound B under strongly basic conditions in anhydrous DMF. The solution is stirred at room temperature for five days after which the solution is evaporated to dryness, re-dissolved in 50 mM triethylammonium acetate buffer (pH 7), and purified with reverse phase HPLC. The ethyl groups are removed from the resulting product compound with trimethyliodosilane and toluidine. The resulting Compound C is purified with reverse phase HPLC:

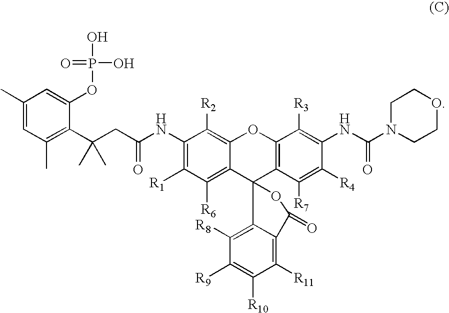

Synthesis of a fluorogenic nucleotide substrate is carried out using the same procedure that is outlined for the generalized conjugation of phosphorylated fluorescein analogs. Briefly, Compound C is reacted with carbonyldiimidazole to generate an imidazole derivative. The resulting product is then conjugated to the nucleotide polyphosphate substrate with an imidazolium ion as a leaving group as in the above protocol.

Synthesis of Self-Generating Coumarin-Based Fluorogenic Nucleotide Substrates (see, e.g., Wang et al., Methods in Molecular Medicine, 1998, 23, 71 and Wang et al., Bioorganic and Medicinal Chemistry Letters, 1996, 6, 945-950)

Coumarin is reacted with LiAlH₄ in anhydrous ethyl ether on ice and then at room temperature for 30 minutes to form Compound D which is chromatographed on silica:

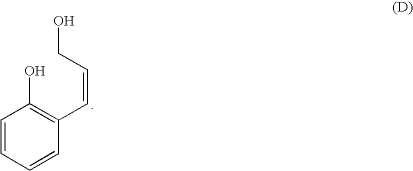

Compound D is then reacted with one equivalent of TBDMS-Cl in anhydrous THF at −5 C followed by the slow addition of a 15-fold molar excess of DMAP. The solution is stirred at −5° C. overnight before being evaporated to dryness and re-dissolved in ethyl acetate. The solution is then washed with hydrochloric acid, sodium bicarbonate, and water, dried, and chromatographed on silica to yield Compound E:

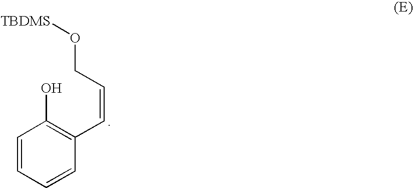

Compound E is combined with a three-fold molar excess of Proton Sponge (Sigma) and co-evaporated with anhydrous DMF. The resulting solid is re-dissolved in DMF under argon, and the solution is cooled to −5° C. A 3-fold molar excess of phosphorous oxychloride is slowly added to the solution which is stirred at −5° C. for 90 minutes before being quenched with 50 mM sodium bicarbonate buffer (pH 8.5). The resulting solution is evaporated to dryness, re-dissolved in 50 mM sodium bicarbonate buffer, and purified with reverse phase HPLC to yield Compound F:

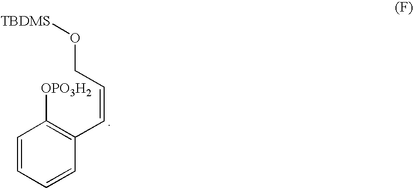

Compound F is treated with acetic acid in THF at room temperature for 4 hours before being evaporated to dryness and purified with reverse phase HPLC to yield Compound G:

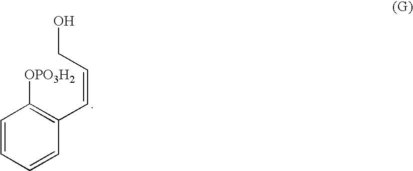

Compound G is treated with manganese (IV) oxide (4-fold molar excess) in anhydrous methylene chloride. This converts the hydroxyl group in Compound G to an aldehyde which is purified on silica gel. The aldehyde is converted to a carboxylic acid by treatment with sodium chlorite in water/acetonitrile followed by 30% hydrogen peroxide at 10° C. for 2.5 hours after which sodium sulfide is added, and the solution is brought to pH 1 with hydrochloric acid. The product is extracted with ethyl acetate and purified with reverse phase HPLC to yield Compound H:

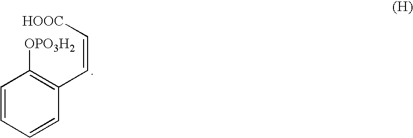

Compound H is co-evaporated with anhydrous DMF, and the resulting solid is re-dissolved in anhydrous DMF under argon. Dicyclohexylcarbodiimide (DCC) is also co-evaporated in anhydrous DMF, re-dissolved in anhydrous DMF, and a 5-fold molar excess of DCC is added to Compound H under argon. After two hours of stirring at room temperature, Compound H is treated with the appropriate amine in the presence of hydroxybenzotriazole and 4-dimethylaminopyridine to form Compound I:

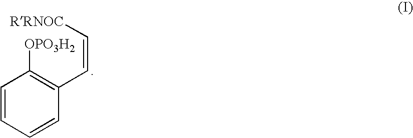

where R and R′ are alkyl groups or variously substituted aromatic rings to form a suitable amine leaving group.

Synthesis of a fluorogenic nucleotide substrate is carried out using the same procedure that is outlined for the generalized conjugation of phosphorylated fluorescein analogs. Briefly, Compound I is reacted with carbonyldiimidazole to generate an imidazole derivative. The resulting product is then conjugated to the nucleotide polyphosphate substrate with an imidazolium ion as a leaving group as in the above protocol to yield:

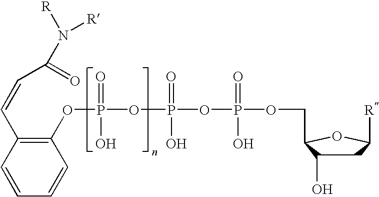

where R″ is a nucleotide base.

### Other Embodiments

All publications, patents, and patent applications mentioned in this specification are herein incorporated by reference to the same extent as if each independent publication or patent application was specifically and individually indicated to be incorporated by reference. While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the appended claims.

Other embodiments are in the claims.

What is claimed is:

1. A method for sequencing a nucleic acid, said method comprising the steps of:
a) disposing in an optionally sealed microreactor a mixture in solution phase comprising a single copy of a target nucleic acid, a nucleic acid replicating catalyst, and a mixture of nucleotides, wherein said mixture of nucleotides comprises a first nucleotide comprising a first label that is substantially non-fluorescent until after incorporation of said first nucleotide into a nucleic acid based on complementarity to said target nucleic acid;
b) allowing continuous template-dependent replication of said target nucleic acid; and
c) sequencing said target nucleic acid by detecting in real time the individual incorporation of said first nucleotide during template-dependent replication by monitoring fluorescence emission resulting from said first label.

2. The method of claim 1, wherein said mixture in solution phase further comprises an activating enzyme that renders said first label fluorescent.

3. The method of claim 2, wherein said activating enzyme is an alkaline phosphatase, acid phosphatase, galactosidase, horseradish peroxidase, phosphodiesterase, phosphotriesterase, pyruvate kinase, lactic dehydrogenase, maltose phosphorylase, glucose oxidase, lipase, or combination thereof.

4. The method of claim 1, wherein said first label is photobleached after step (c).

5. The method of claim 1, wherein said first label is a phosphate label that is cleaved from said first nucleotide during replication.

6. The method of claim 1, wherein said mixture of nucleotides further comprises a second nucleotide comprising a second label that is substantially non-fluorescent until incorporation of said second nucleotide into said nucleic acid based on complementarity to said target nucleic acid.

7. The method of claim 6, wherein said mixture of nucleotides further comprises a third nucleotide comprising a third label that is substantially non-fluorescent until incorporation of said third nucleotide into said nucleic acid based on complementarity to said target nucleic acid.

8. The method of claim 7, wherein said mixture of nucleotides further comprises a fourth nucleotide comprising a fourth label that is substantially non-fluorescent until incorporation of said fourth nucleotide into said nucleic acid based on complementarity to said target nucleic acid.

9. The method of claim 1, further comprising repeating steps (b)-(c) at least once.

10. The method of claim 1, wherein said mixture in a solution phase has a volume of 0.0001 fL-1000 fL.

11. The method of claim 1, wherein said nucleic acid replicating catalyst is DNA polymerase, RNA polymerase, ligase, reverse transcriptase, or RNA-dependent RNA polymerase.

12. The method of claim 1, wherein said target nucleic acid is DNA, and said mixture in solution phase further comprises a primer.

13. The method of claim 1, wherein said target nucleic acid is RNA.

14. The method of claim 1, wherein steps (a)-(c) are repeated to obtain the sequence for 10, 25, 100, 300, 1000, or 10,000 base pairs of said target nucleic acid.

15. The method of claim 1, wherein said sequencing occurs continuously.

16. The method of claim 1, wherein said nucleic acid is immobilized on a surface of said microreactor.

17. The method of claim 1, wherein said nucleic acid is immobilized on a bead disposed in said microreactor.

18. The method of claim 2, wherein said activating enzyme is immobilized on a surface of said microreactor.

19. The method of claim 2, wherein said nucleic acid is immobilized on a bead disposed in said microreactor.

20. A compound having the formula:
Base-Sugar-Phosphate-[Self-reacting Component],
where Base is a nucleotide base, Sugar is selected from the group consisting of ribose, 2′-deoxyribose, 2′-O-methyl-ribose, ribose comprising a methylene connecting the 2′ oxygen and 4′ carbon, glycerol, 2-methyl morpholine, or threose, Phosphate is a polyphosphate, and Self-reacting Component is a moiety that undergoes an intramolecular reaction upon cleavage of the phosphate to which it is connected to form a fluorophore.

21. The compound of claim 20, wherein Sugar is ribose or 2′-deoxyribose.

22. The compound of claim 20, wherein Base is cytosine, guanine, adenine, thymine, uracil, xanthine, hypoxanthine, inosine, orotate, thioinosine, thiouracil, pseudouracil, 5,6-dihydrouracil, and 5-bromouracil.

23. The compound of claim 20, wherein Phosphate is a triphosphate.

24. The compound of claim 20, wherein [Self-reacting Component] comprises a self-immolative linker.

25. The compound of claim 20, wherein [Self-reacting Component] comprises a moiety that undergoes an intramolecular reaction to form a fluorophore upon removal of the phosphate.

26. The compound of claim 20, having the formula: 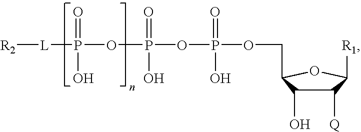
wherein Q is H, OH, or OMe, n is an integer from 1 to 4; R₁ is cytosine, guanine, adenine, thymine, or uracil; L is a self-immolative linker; and R₂ is a fluorophore bound to said linker via an amine group.

27. The compound of claim 24, wherein said self-immolative linker is 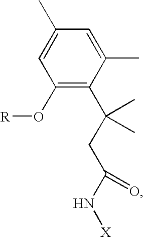
wherein R is Phosphate; and X—NH is a fluorophore bound to said linker via an amine group.

28. The compound of claim 27, wherein X—NH has the formula 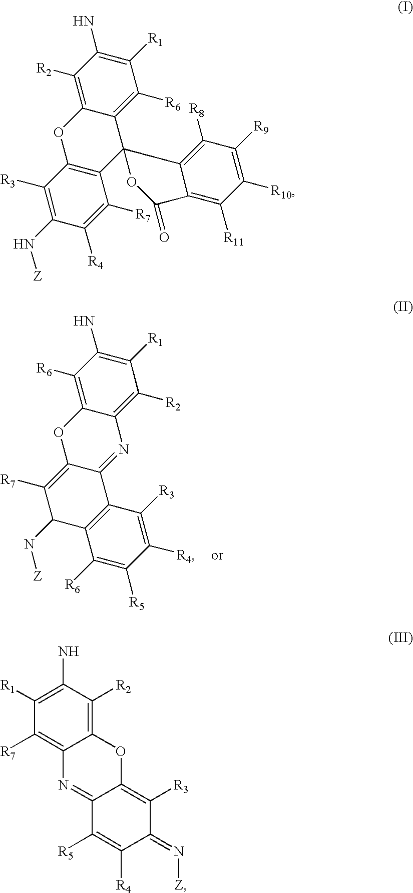
wherein each of R₁-R₁₁ is independently selected from hydrogen, halogen, sulfonate, carboxy, C₁₋₆ acyl, or C₁₋₆ alkyl, C₁₋₆ alkoxy, C₁₋₆ alkylthio, a C₁₋₆ alkyl group interrupted with one or more heteroatoms, C₁₋₆ haloalkyl group, C₃₋₆ cycloalkyl, carboxy substituted C₁₋₆ alkyl, carboxy substituted C₁₋₆ alkoxy, carboxy substituted C₁₋₆ alkylthio, C₆₋₁₀ aryl, C₄₋₉ heteroaryl, nitro, sulfonyl substituted C₁₋₆ alkyl, or hydroxyl, and each Z is independently C₁₋₆ acyl, C₁₋₆ alkyl, sulfonyl, a C₁₋₆ alkyl group interrupted with one or more heteroatoms, C₁₋₆ haloalkyl group, C₃₋₆ cycloalkyl, carboxy substituted C₁₋₆ alkyl, or sulfonyl substituted C₁₋₆ alkyl.

29. The compound of claim 25, having the formula: 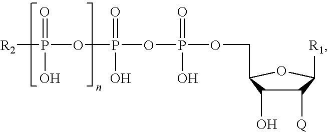
where Q is H, OH, or OMe, n is an integer from 1 to 4; R₁ is cytosine, guanine, adenine, thymine, or uracil; and R₂ is said moiety that undergoes an intramolecular reaction to form a fluorophore upon removal of the phosphate.

30. The compound of claim 25, wherein said moiety that undergoes an intramolecular reaction to form a fluorophore upon removal of the phosphate has the formula: 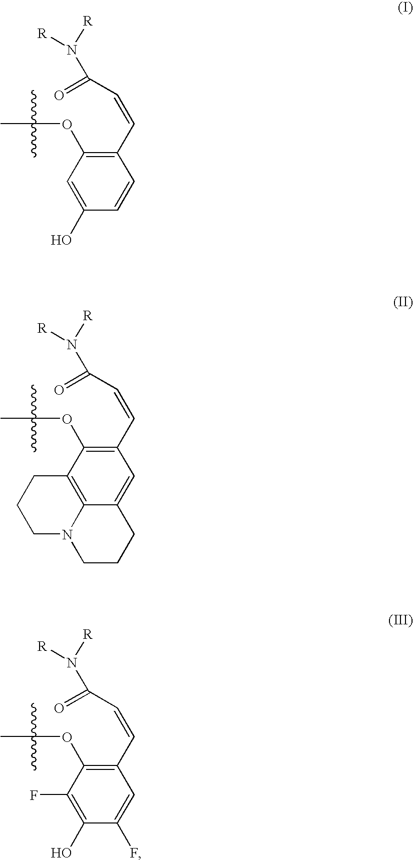
wherein each R is independently H or C₁₋₆ alkyl, or both R groups together are C₂₋₅ alkylene.

31. A compound having the formula: 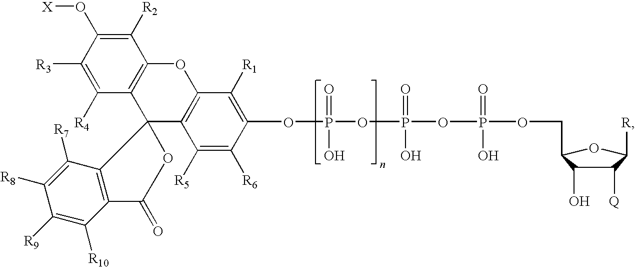
where R is a nucleotide base, Q is H, OH, or OMe, n is an integer from 1 to 4, and R₁-R₁₀ are independently selected from hydrogen, halogen, sulfonate, carboxy, C₁₋₆ acyl, or C₁₋₆ alkyl, C₁₋₆ alkoxy, C₁₋₆ alkylthio, a C₁₋₆ alkyl group interrupted with one or more heteroatoms, C₁₋₆ haloalkyl group, C₃₋₆ cycloalkyl, carboxy substituted C₁₋₆ alkyl, carboxy substituted C₁₋₆ alkoxy, carboxy substituted C₁₋₆ alkylthio, C₆₋₁₀ aryl, C₄₋₉ heteroaryl, nitro, sulfonyl substituted C₁₋₆ alkyl, or hydroxyl, and X is C₁₋₆ acyl, C₁₋₆ alkyl, sulfonyl, a C₁₋₆ alkyl group interrupted with one or more heteroatoms, C₁₋₆ haloalkyl group, C₃₋₆ cycloalkyl, carboxy substituted C₁₋₆ alkyl, or sulfonyl substituted C₁₋₆ alkyl, wherein when R₁-R₁₀ are H, X is not ethyl.
The compound of claim 31, having the formula:

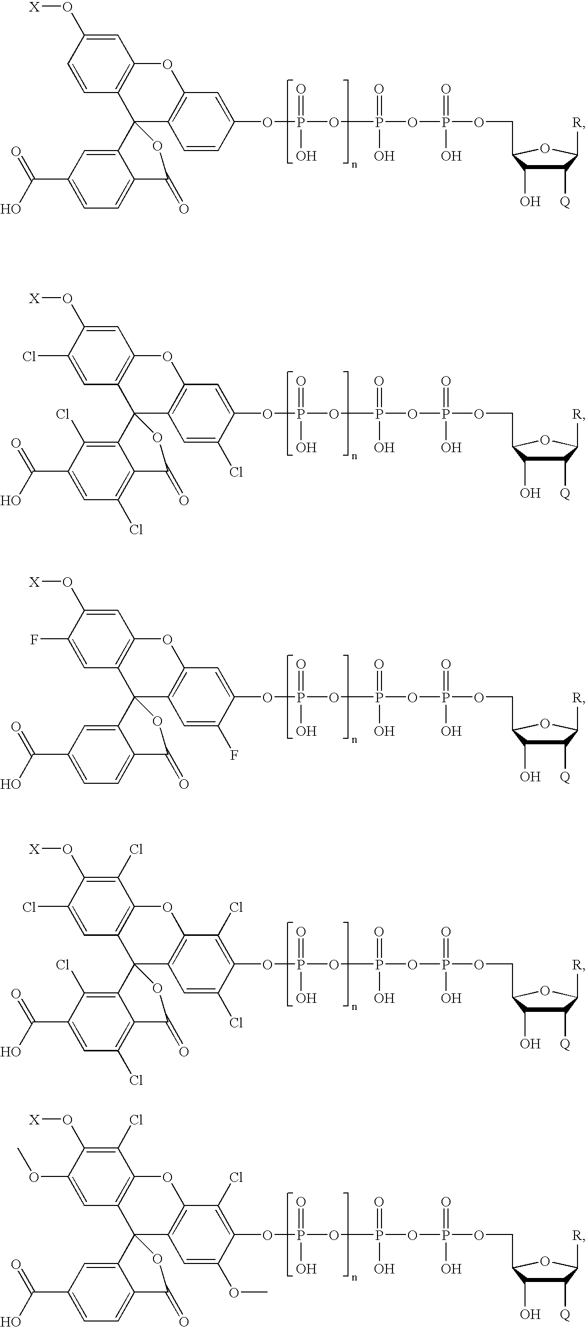

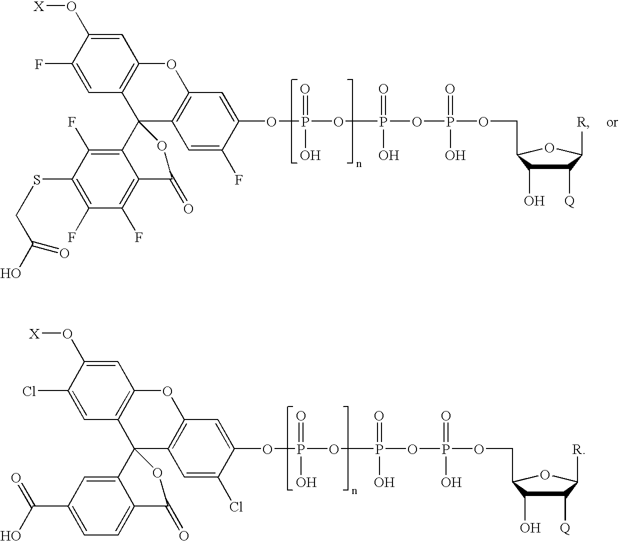

32. The compound of claim 31, wherein R is cytosine, guanine, adenine, thymine, uracil, xanthine, hypoxanthine, inosine, orotate, thioinosine, thiouracil, pseudouracil, 5,6-dihydrouracil, and 5-bromouracil.

33. A compound having the formula: 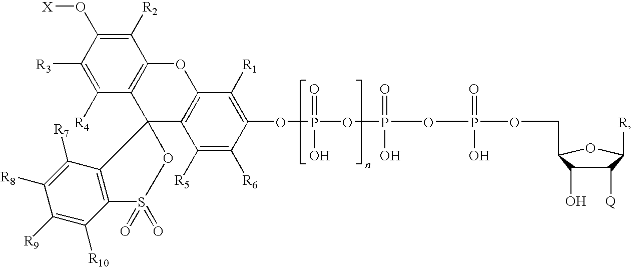
wherein R is a nucleotide base, Q is H, OH, or OMe, n is an integer from 1 to 4, and R₁-R₁₀ are independently selected from hydrogen, halogen, sulfonate, carboxy, C₁₋₆ acyl, or C₁₋₆ alkyl, C₁₋₆ alkoxy, C₁₋₆ alkylthio, a C₁₋₆ alkyl group interrupted with one or more heteroatoms, C₁₋₆ haloalkyl group, C₃₋₆ cycloalkyl, carboxy substituted C₁₋₆ alkyl, carboxy substituted C₁₋₆ alkoxy, carboxy substituted C₁₋₆ alkylthio, C₆₋₁₀ aryl, C₄₋₉ heteroaryl, nitro, sulfonyl substituted C₁₋₆ alkyl, or hydroxyl, and X is C₁₋₆ acyl, C₁₋₆ alkyl, sulfonyl, a C₁₋₆ alkyl group interrupted with one or more heteroatoms, C₁₋₆ haloalkyl group, C₃₋₆ cycloalkyl, carboxy substituted C₁₋₆ alkyl, or sulfonyl substituted C₁₋₆ alkyl.

34. The compound of claim 33, wherein R is cytosine, guanine, adenine, thymine, uracil, xanthine, hypoxanthine, inosine, orotate, thioinosine, thiouracil, pseudouracil, 5,6-dihydrouracil, and 5-bromouracil.

35. The compound of claim 33, having the formula: [chemical structure]
